(19) United States
(12) Patent Application Publication
(10) Pub. No.: US 2026/0325009 A1
(43) Pub. Date: Oct. 22, 2026

(54) WEARABLE ELECTRONIC DEVICE AND METHOD FOR DETERMINING STATE OF BEING WORN ON HUMAN BODY

(71) Applicant: Samsung Electronics Co., Ltd., Suwon-si (KR)

(72) Inventors: Yongsang CHO, Suwon-si (KR); Wonsuk Chung, Suwon-si (KR); Cheolo Kim, Suwon-si (KR); Jinyoup Ahn, Suwon-si (KR)

(73) Assignee: Samsung Electronics Co., Ltd., Suwon-si (KR)

(21) Appl. No.: 19/536,965

(22) Filed: Feb. 11, 2026

Related U.S. Application Data
(63) Continuation of application No. PCT/KR2026/000608, filed on Jan. 12, 2026.

(30) Foreign Application Priority Data
Jan. 31, 2025 (KR) 10-2025-0012307
Mar. 14, 2025 (KR) 10-2025-0033615

(51) Int. Cl. A61B 5/11; A61B 5/00

(52) U.S. Cl. CPC A61B 5/1116; A61B 5/486; A61B 5/6802; A61B 2560/0223

(57) ABSTRACT

An electronic device capable of being worn on a finger includes a motion sensor configured to detect movement of the electronic device, a processor, and a memory configured to store instructions. The instructions may be configured to cause, when executed by the processor, the electronic device to generate a first message requesting a user to perform a specified first gesture, obtain, at least based on the generation of the first message, a first vector and a second vector indicating acceleration in a direction of gravity using data generated by the motion sensor, determine a first x-axis acceleration value in an x-axis direction, a first y-axis acceleration value in a y-axis direction, and a first z-axis acceleration value in a z-axis direction using the first vector and the second vector, and calculate a correction value to convert the first x-axis acceleration value, the first y-axis acceleration value, and the first z-axis acceleration value into a second x-axis acceleration value, a second y-axis acceleration value, and a second z-axis acceleration value measured when the electronic device is in a specified wearing state.

### CROSS-REFERENCE TO RELATED APPLICATIONS

This application is a continuation of International Application No. PCT/KR2026/000608, filed on Jan. 12, 2026, which is based on and claims priority to Korean Patent Application No. 10-2025-0012307, filed on Jan. 31, 2025, in the Korean Intellectual Property Office, and to Korean Patent Application No. 10-2025-0033615, filed on Mar. 14, 2025, in the Korean Intellectual Property Office the disclosures of which are incorporated by reference herein in their entireties.

### BACKGROUND

### 1. Field

The disclosure relates to a wearable electronic device for determining the state of the wearable electronic device being worn on a human body, in particular, a finger, and a method therefor.

### 2. Description of Related Art

Wearable electronic devices may include a sensor (e.g., an inertial sensor) for detecting a user's gesture and a communication circuit (e.g., a Bluetooth communication circuit) for wirelessly communicating with another electronic device (e.g., a smartphone). The electronic device may identify a specified movement pattern from raw data generated by the sensor and transmit an event (or a control signal) corresponding to the movement pattern to another electronic device (e.g., a smartphone) through a wireless communication circuit. The electronic device that receives the event may perform a function specified for the event. For example, the electronic device may take a photo or make a phone call in response to the reception of the event.

The above-described information is provided as related art for the purpose of assist in understanding the disclosure No assertion or decision is made as to whether any of the above might be applicable as prior art with regard to the disclosure.

### SUMMARY

Wearable electronic devices may include head-mounted wearable electronic devices (hereinafter, head-mounted display (HMD) devices), wrist-mounted wearable electronic devices (hereinafter, watch-type devices), and finger-mounted wearable electronic devices (hereinafter, ring-type devices).

When the watch-type device is worn on the wrist, the position of a sensor may be fixed relative to the wrist in the watch-type device. In the HMD device, the position of a sensor may also be fixed relative to the head. On the other hand, the position of a sensor in the ring-type device may vary each time it is worn on the finger. In addition, the ring-type device may be rotated on the finger, so that the position of a sensor may sometimes change even while it is worn. When the position of the sensor changes, the data generated from the sensor may vary even if the user makes substantially the same movement pattern (e.g., a user gesture of shaking the hand up and down). Therefore, the ring-type device may need to recognize the state of the device being worn on a finger and correct sensor data, based on the recognized state, before performing an operation of identifying whether there is a specified movement pattern in the sensor data. Correction of the sensor data may enable the movement pattern to be accurately identified.

In various embodiments of the disclosure, the ring-type device is capable of accurately recognizing the state (or posture) of being worn on a finger. In the disclosure, the state may be defined by the direction in which the sensor faces. For example, the sensor may include an inertial sensor for detecting acceleration and determining the direction in which gravity acts. For example, the inertial sensor may be configured to generate a first acceleration value corresponding to motion in the x-axis direction, a second acceleration value corresponding to motion in the y-axis direction, and a third acceleration value corresponding to motion in the z-axis direction. The ring-type device may determine the wearing state using data (e.g., data representing acceleration values of respective axes and data representing the direction of gravity) obtained through the sensor.

The technical subjects pursued in the disclosure may not be limited to the above-mentioned technical subjects, and other technical subjects which are not mentioned herein may be clearly understood from the following description by those skilled in the art to which the disclosure pertains.

According to an embodiment, an electronic device capable of being worn on a finger includes a motion sensor configured to detect movement of the electronic device, a processor, and a memory configured to store instructions. The instructions, when executed by the processor, may cause the electronic device to generate a first message requesting a user to perform a specified first gesture. The instructions may cause the electronic device to obtain, at least based on the generation of the first message, a first vector and a second vector indicating acceleration in the direction of gravity using data generated by the motion sensor. The instructions may cause the electronic device to determine a first x-axis acceleration value in an x-axis direction, a first y-axis acceleration value in a y-axis direction, and a first z-axis acceleration value in a z-axis direction using the first vector and the second vector. The instructions may cause the electronic device to calculate a correction value to convert the first x-axis acceleration value, the first y-axis acceleration value, and the first z-axis acceleration value into a second x-axis acceleration value, a second y-axis acceleration value, and a second z-axis acceleration value measured when the electronic device is in a specified wearing state.

According to an embodiment, there is provided a recording medium storing instructions readable by a processor of an electronic device capable of being worn on a finger. The instructions, when executed by the processor, may cause the electronic device to generate a first message requesting a user to perform a specified first gesture. The instructions may cause the electronic device to obtain, at least based on the generation of the first message, a first vector and a second vector indicating acceleration in the direction of gravity using data generated by a motion sensor of the electronic device. The instructions may cause the electronic device to determine a first x-axis acceleration value in an x-axis direction, a first y-axis acceleration value in a y-axis direction, and a first z-axis acceleration value in a z-axis direction using the first vector and the second vector. The instructions may cause the electronic device to calculate a correction value to convert the first x-axis acceleration value, the first y-axis acceleration value, and the first z-axis acceleration value into a second x-axis acceleration value, a second y-axis acceleration value, and a second z-axis acceleration value measured when the electronic device is in a specified wearing state.

According to an embodiment, an electronic device capable of being worn on a finger includes a communication circuit for the electronic device to wirelessly communicate with an external electronic device, a motion sensor configured to detect movement of the electronic device, a processor, and a memory configured to store instructions, wherein the instructions may be configured to cause, when executed by the processor, the electronic device to periodically identify a user's state using data generated by the motion sensor. The instructions may cause the electronic device to identify, based on the user's state being identified as a walking or running state, a first direction in which a user wearing the electronic device moves forward using data generated by the motion sensor. The instructions may cause the electronic device to identify that the electronic device moves backward in a second direction opposite the first direction using data generated by the motion sensor, and obtain a third acceleration value at the time of moving backward. The instructions may cause the electronic device to identify that the electronic device moves forward in the first direction using data generated by the motion sensor, and obtain a fourth acceleration value at the time of moving forward. The instructions may cause the electronic device to determine a first x-axis acceleration value in an x-axis direction, a first y-axis acceleration value in a y-axis direction, and a first z-axis acceleration value in a z-axis direction using the third acceleration value and the fourth acceleration value. The instructions may cause the electronic device to calculate a correction value to convert the first x-axis acceleration value, the first y-axis acceleration value, and the first z-axis acceleration value into a second x-axis acceleration value, a second y-axis acceleration value, and a second z-axis acceleration value measured when the electronic device is in a specified wearing state.

According to an embodiment, there is provided a method for operating an electronic device capable of being worn on a finger. The method may include generating a first message requesting a user to perform a specified first gesture. The method may include obtaining, at least based on the generation of the first message, a first vector and a second vector indicating acceleration in a direction of gravity using data generated by a motion sensor of the electronic device. The method may include determining a first x-axis acceleration value in an x-axis direction, a first y-axis acceleration value in a y-axis direction, and a first z-axis acceleration value in a z-axis direction using the first vector and the second vector. The method may include calculating a correction value to convert the first x-axis acceleration value, the first y-axis acceleration value, and the first z-axis acceleration value into a second x-axis acceleration value, a second y-axis acceleration value, and a second z-axis acceleration value measured when the electronic device is in a specified wearing state.

An embodiment of the disclosure may provide a ring-type device configured to accurately recognize the state (or posture) of the device being worn on a finger. Various other effects understood directly or indirectly through the disclosure may be provided.

### BRIEF DESCRIPTION OF THE DRAWINGS

FIG. 1 is a block diagram of an electronic device in a network environment according to various embodiments.

FIG. 2A is a perspective view of a wearable electronic device according to an embodiment. FIG. 2B is a front view of the wearable electronic device shown in FIG. 2A.

FIG. 2C is an exploded perspective view of the wearable electronic device shown in FIG. 2A.

FIG. 3 illustrates a configuration of a system for determining a state of being worn on a human body according to an embodiment.

FIG. 4A illustrates an acceleration sensor applicable as the motion sensor in FIG. 3.

FIG. 4B illustrates a first acceleration value when a hand moves in a first direction while a ring-type device is worn on a finger in a first state.

FIG. 4C illustrates a second acceleration value when a hand moves in a first direction while a ring-type device is worn on a finger in a second state.

FIGS. 5A, 5B, 5C, and 5D are drawings illustrating an operation for guiding a user to make a specified gesture and calculating a correction value according to an embodiment.

FIGS. 6A and 6B and FIGS. 7A and 7B illustrate cases of correcting acceleration values using the embodiments described with reference to FIGS. 5A, 5B and 5C.

FIG. 8 is a drawing illustrating an operation for calculating a correction value, based on a change in linear acceleration, according to an embodiment.

FIGS. 9A and 9B and FIGS. 10A and 10B illustrate cases of correcting acceleration values using the embodiments described with reference to FIG. 8.

FIG. 11 is a drawing illustrating an operation for calculating a correction value, based on a result of monitoring a user's state, according to an embodiment.

FIG. 12 is a flowchart illustrating an overall operation of a ring-type device to generate an event in response to a user's gesture according to an embodiment.

FIG. 13 is a flowchart illustrating an operation of a ring-type device to obtain a correction value according to an embodiment.

FIGS. 14A and 14B illustrate examples of user interface (UI) screens that may be presented to a user regarding a gesture that is to be performed by the user.

FIG. 15 is a flowchart illustrating an operation of a ring-type device for obtaining a correction value according to an embodiment.

FIG. 16 illustrates an example of a UI screen that may be presented to a user regarding a gesture that is to be performed by the user.

FIG. 17 illustrates an example of a UI screen that allows a user to configure whether to automatically perform correction.

FIG. 18 is a flowchart illustrating an operation of a ring-type device for obtaining a correction value according to an embodiment.

Hereinafter, embodiments of the disclosure will be described in detail with reference to the drawings so that those skilled in the art to which the disclosure pertains can easily implement the disclosure. However, the disclosure may be implemented in various different forms and is not limited to embodiments set forth herein. With regard to the description of the drawings, the same or like reference signs may be used to designate the same or like elements. Also, in the drawings and the relevant descriptions, description of well-known functions and configurations may be omitted for the sake of clarity and brevity.

### DETAILED DESCRIPTION

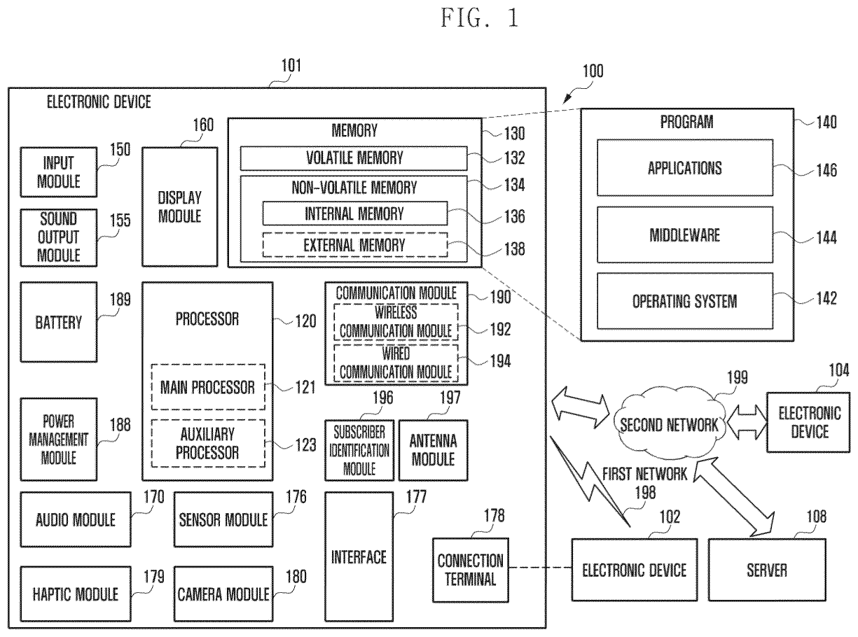

FIG. 1 is a block diagram illustrating an electronic device 101 in a network environment 100 according to various embodiments. Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module(SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to one embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thererto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to one embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (QEC), or client-server computing technology may be used, for example. The electronic device 101 may provide ultra low-latency services using, e.g., distributed computing or mobile edge computing. In another embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

One or more processors 120 may be provided. For example, the processor 120 may have a multi-core processor structure such as a dual-core, a quad-core, or a hexa-core.

The processor 120 may control the operations of the electronic device 101 by executing instructions stored in the memory 130. For example, the processor 120 may correspond to a plurality of processors collectively performing a plurality of operations by dividing them across the processors.

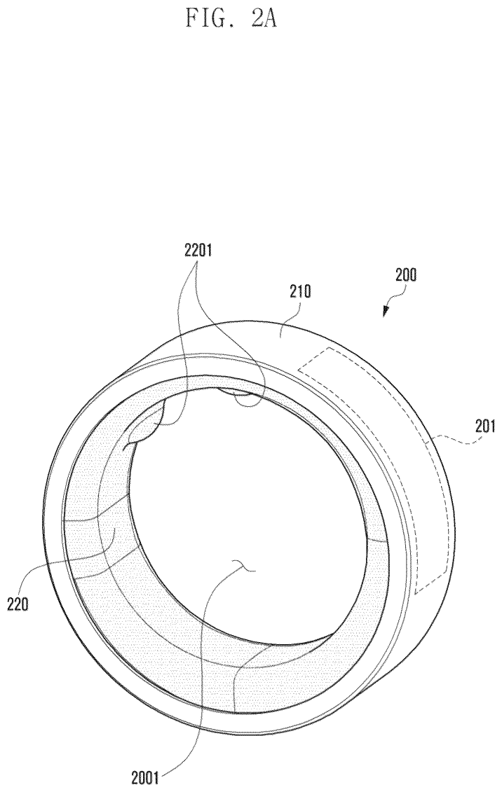

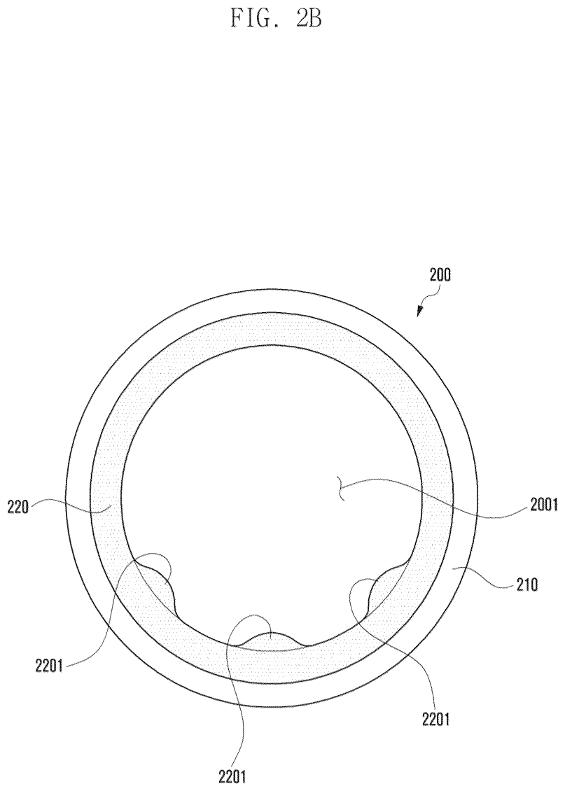

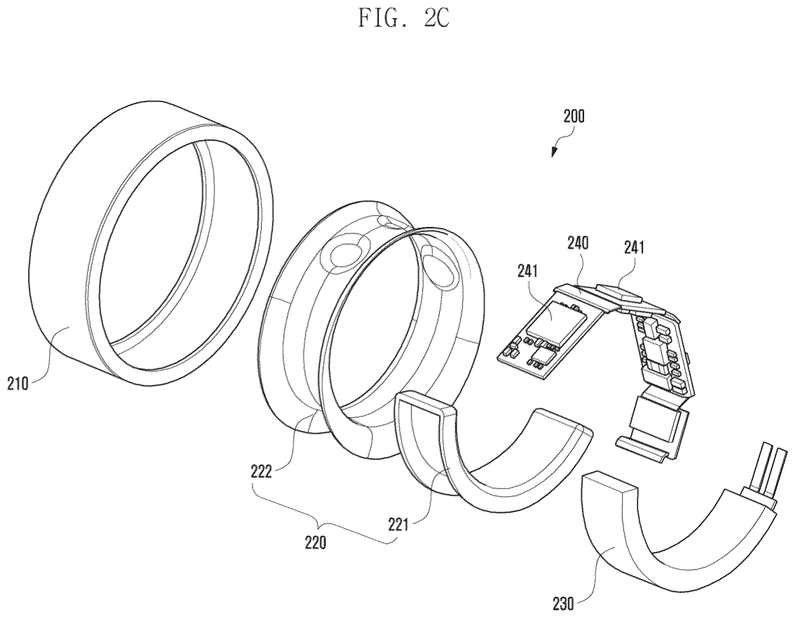

FIG. 2A is a perspective view of a wearable electronic device according to an embodiment. FIG. 2B is a front view of the wearable electronic device shown in FIG. 2A. FIG. 2C is an exploded perspective view of the wearable electronic device shown in FIG. 2A. Although a ring-type wearable electronic device 200 to be worn on a user's finger will be illustrated and described herein, the disclosure is not limited thereto. It is obvious to those skilled in the art that the embodiments of the disclosure are able to be applied to, for example, a bracelet-type wearable electronic device, an open ring-type electronic device having an open portion, or a curved or non-curved electronic device.

Referring to FIGS. 2A and 2B, the wearable electronic device 200 may be formed in an annular shape that includes an opening 2001 therein. In an embodiment, the wearable electronic device 200 may include an annular first housing 210 (e.g., an outer ring housing, a first ring housing, or a first housing portion), and an annular second housing 220 (e.g., an inner ring housing, a second ring housing, or a second housing portion) coupled to the first housing 210 and including the opening 2001. In an embodiment, the opening 2001 may be formed in a size enabling the user's finger to be inserted thereinto. In an embodiment, the wearable electronic device 200 may include at least one protrusion 2201 protruding from the second housing 220 toward the opening 2001. In an embodiment, at least one protrusion 2201 may be disposed in the inner space of the wearable electronic device 200, and may have a shape that is advantageous for detecting an external environment or making contact with the user's skin. In some embodiments, at least one protrusion 2201 may be used as a means for preventing the wearable electronic device 200 from being randomly rotated on a finger.

According to an embodiment, the wearable electronic device 200 may include at least one electrical component disposed in the space between the first housing 210 and the second housing 220. In an embodiment, at least one electrical component may include at least one biometric sensor disposed to detect biometric information of the user through at least a portion of the second housing 220, a substrate (e.g., the substrate 240 in FIG. 2C), a display 201, or an output module. In an embodiment, the substrate (e.g., the substrate 240 in FIG. 2C) may include a flexible printed circuit board (FPCB) that is bendable to correspond to the curvature of the wearable electronic device 200. In an embodiment, the display 201 may be disposed so as to be visible from the outside through a portion (e.g., outer surface) of the first housing 210. In an embodiment, the wearable electronic device 200 may further include an indicator, such as a light-emitting diode (LED), that may provide visual output information to the user. In some embodiments, the indicator may replace the display 201. In an embodiment, the display 201 may be formed on the entire front surface of the first housing 210. In an embodiment, the output module may include at least one speaker (not shown) for providing auditory output information to the user. In an embodiment, the output module may include a haptic module for providing tactile output information to the user.

Referring to FIG. 2C, the wearable electronic device 200 may include a first housing 210, a second housing 220 coupled to the first housing 210, and a battery 230 disposed between the first housing 210 and the second housing 220. In an embodiment, the wearable electronic device 200 may include a substrate 240 disposed between the first housing 210 and the second housing 220 and including a plurality of electrical elements 241. In an embodiment, the substrate 240 may include a flexible printed circuit board (FPCB) that is bendable to correspond to the curvature of the wearable electronic device 200. In some embodiments, the substrate 240 may include a substrate including a hard-type region having a width and length, which is not affected by the curvature of the first housing 210 and/or the second housing 220, or a plurality of hard-type substrates (printed circuit boards (PCBs)). In an embodiment, the battery 230 may be disposed between the first housing 210 and the second housing 220 so as to be spaced apart from the substrate 240 by a predetermined distance, and may be electrically connected to the substrate 240 through a cable. In an embodiment, the battery 230 may be formed in a curved shape to have a curvature substantially identical to the curvature of the first housing 210. In some embodiments, the battery 230 may be disposed between the first housing 210 and the second housing 220 to have a curvature different from the curvature of the first housing 210 and/or the curvature of the second housing 220.

According to an embodiment, the first housing 210 may be formed of a metal material, a ceramic material, or a PC material. In an embodiment, the second housing 220 may be coupled to the first housing 210 through a molding process using a molding material. In an embodiment, the second housing 220 may include a first molding layer 221 disposed to cover at least a portion of the battery 230, and a second molding layer 222 covering the first molding layer 221 and coupled to the first housing 210. In an embodiment, the first molding layer 221 may be disposed so as to cover the entirety of the battery 230 and be in contact, at least in part, with the inner surface of the first housing 210.

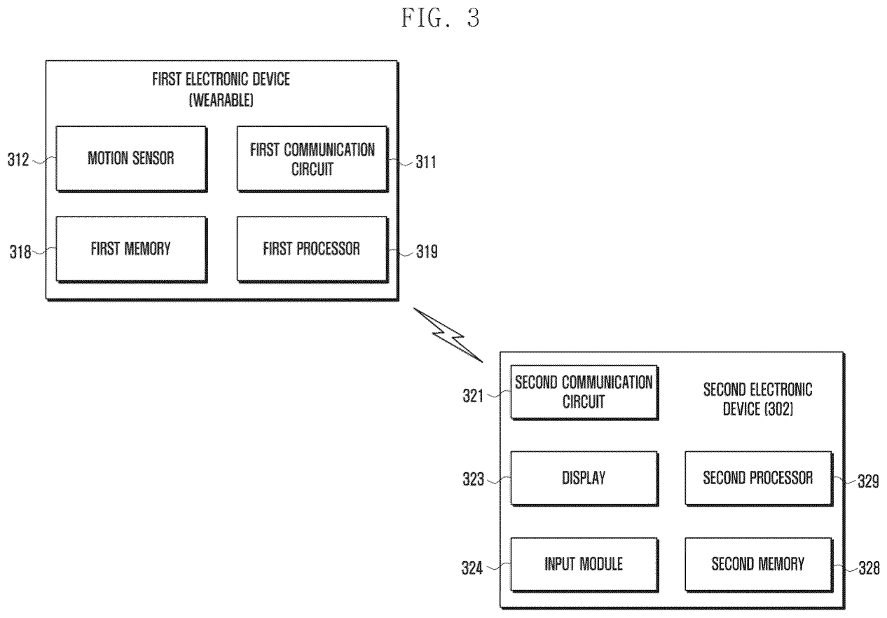

FIG. 3 illustrates a configuration of a system for determining a state of being worn on a human body according to an embodiment. The system may include a first electronic device 301 and a second electronic device 302. The first electronic device 301 may be a device (e.g., the wearable electronic device 200 in FIG. 2A) capable of being worn on the user's body. The second electronic device 302 may be a device capable of short-range wireless communication (e.g., Bluetooth or Wi-Fi) with the first electronic device 301 and capable of displaying information. The second electronic device 302 may be a portable electronic device (e.g., a smartphone) or a wearable electronic device capable of being worn on the human body (e.g., a wrist or head).

The first electronic device 301 may include a first communication circuit 311, a motion sensor 312, a first memory 318, and a first processor 319. The first communication circuit 311, the first memory 318, and the first processor 319 may be respectively implemented to be substantially identical to the communication module 190, the memory 130, and the first processor 120 in FIG. 1, thereby performing the same functions. The first communication circuit 311, the motion sensor 312, the first memory 318, and the first processor 319 may be disposed on a substrate (e.g., the substrate 240 in FIG. 2C). The first electronic device 301 may further include a display (e.g., the display 201 in FIG. 2A).

The motion sensor 312 may be configured to generate raw data used to recognize the direction in which the first electronic device 301 moves and the magnitude (e.g., acceleration) of the movement. For example, the motion sensor 312 may include an inertia measurement unit (IM) configured to include an acceleration sensor, a gyroscope (in other words, an angular velocity sensor), and a magnetometer. The acceleration sensor in the motion sensor 312 may be configured to generate a first acceleration value corresponding to the movement of the first electronic device 301 in the x-axis direction, a second acceleration value corresponding to the movement of the first electronic device 301 in the y-axis direction, and a third acceleration value corresponding to the movement of the first electronic device 301 in the z-axis direction. The gyroscope in the motion sensor 312 may detect an angular velocity due to rotation. The gyroscope may utilize the characteristics of an object having a tendency to maintain a constant orientation in space while rotating. For example, the gyroscope may measure rotation by sensing a Coriolis force effect that occurs according to the rotation speed. The gyroscope may be configured to generate a first angular velocity value corresponding to the rotation of the first electronic device 301 about the x-axis, the second angular velocity value corresponding to the rotation of the first electronic device 301 about the y-axis, and the third angular velocity value corresponding to the rotation of the first electronic device 301 about the z-axis. The first processor 319 may calculate Euler angles (roll, pitch, and yaw) using data (e.g., data indicating acceleration values of respective axes and data indicating the direction in which the Earth's magnetic force is directed) received from the motion sensor 312.

The motion sensor 312 (e.g., an acceleration sensor) may generate raw data used to recognize the direction in which gravity acts. The first processor 310 may generate a vector indicating acceleration (gravity acceleration) in the direction of gravity using the raw data obtained from the motion sensor 312.

Instructions may be stored in the memory of the first electronic device 301. The memory may include a first memory 318 and/or an internal memory of the first processor 319. For example, instructions may be stored in the first memory 318 or the internal memory of the first processor 319. As another example, instructions may be partially stored in the first memory 318 and the internal memory of the first processor 319. Instructions may cause, when executed by the first processor 319, the first electronic device 301 to perform the operation, which will be described later with reference to the drawings.

According to an embodiment, the second electronic device 302 may include a second communication circuit 321, a display 323, an input module 324, a second memory 328, and a second processor 329. The second communication circuit 321, the display 323, the input module 324, the second memory 328, and the second processor 329 may be respectively implemented to be substantially identical to the communication module 190, the display module 160, the input module 150, the memory 130, and the first processor 120 in FIG. 1, thereby performing the same functions.

Instructions may be stored in a memory of the second electronic device 302. The memory may include a second memory 328 and/or an internal memory of the second processor 329. For example, instructions may be stored in the second memory 328 or the internal memory of the second processor 329. As another example, instructions may be partially stored in the second memory 328 and the internal memory of the second processor 329. Instructions may cause, when executed by the second processor 329, the second electronic device 302 to perform the operation, which will be described later with reference to the drawings.

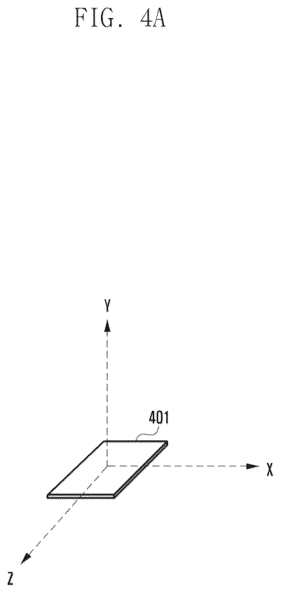

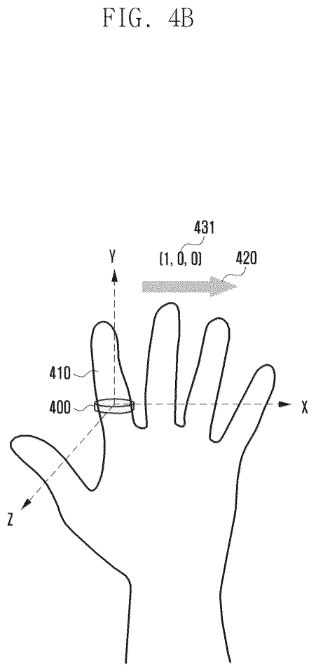

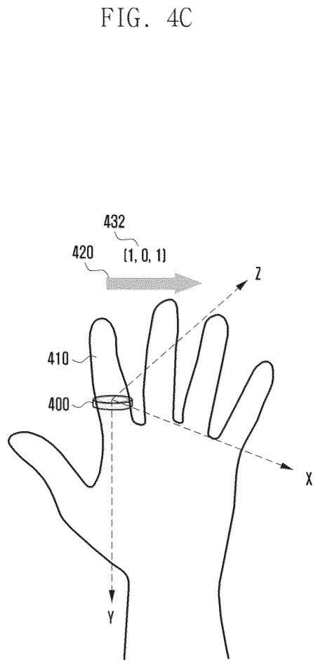

FIG. 4A illustrates an acceleration sensor 401 applicable as the motion sensor 312 in FIG. 3. FIG. 4B illustrates a first acceleration value 431 calculated by a ring-type device 400 (e.g., the first electronic device 301 in FIG. 3) having an acceleration sensor 401 when a hand moves in a first direction 420 while the ring-type device 400 is worn on a finger 410 in a first state. FIG. 4C illustrates a second acceleration value 432 calculated by a ring-type device 400 when a hand moves in the same first direction 420 while the ring-type device 400 is worn on a finger 410 in a second state, which is different from the first state.

Referring to FIG. 4A, the acceleration sensor 401 may generate raw data indicating acceleration in the X-axis direction in the acceleration sensor 401, raw data indicating acceleration in the Y-axis direction in the acceleration sensor 401, and raw data indicating acceleration in the Z-axis direction in the acceleration sensor 401. The ring-type device 400 may obtain acceleration values (x, y, z) using raw data generated from the acceleration sensor 401. Here, x, y, or z indicates the magnitude of acceleration in the corresponding direction.

Referring to FIG. 4B, the ring-type device 400 may be worn on a finger 410 in a first state. For example, the direction in which the X-axis of the acceleration sensor 401 is directed may be the same as the first direction 420. Accordingly, the first acceleration value 431 obtained in the first state may be (1, 0, 0).

Referring to FIG. 4C, the ring-type device 400 may be worn on the finger 410 in a second state. For example, the directions in which the X-axis and Z-axis of the acceleration sensor 401 are directed may be oblique to the first direction 420. Accordingly, the second acceleration value 432 obtained in the second state may be (1, 0, 1). Although the hand moves in the same direction, since the wearing states are different, the second acceleration value 432 is different from the first acceleration value 431. Therefore, the acceleration value obtained using the acceleration sensor 401 is required to be corrected in order to recognize substantially the same gesture (e.g., hand movement in the first direction 420). According to various embodiments of this document, the electronic device (e.g., the first electronic device 301) may calculate a correction value to correct the acceleration value obtained using the motion sensor and, by such correction (which could also be considered a recognition of the electronic device of its relative position), calibrate the electronic device relative to a worn position of the device. Such corrections may be understood to be calibrations herein according to one or more embodiments.

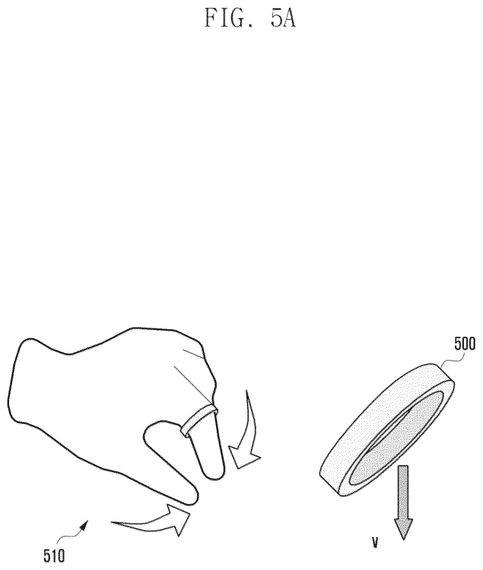

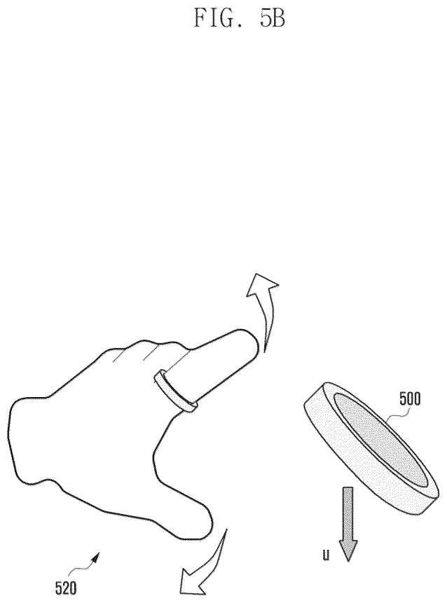

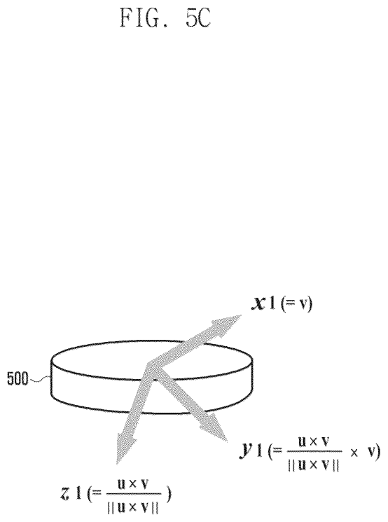

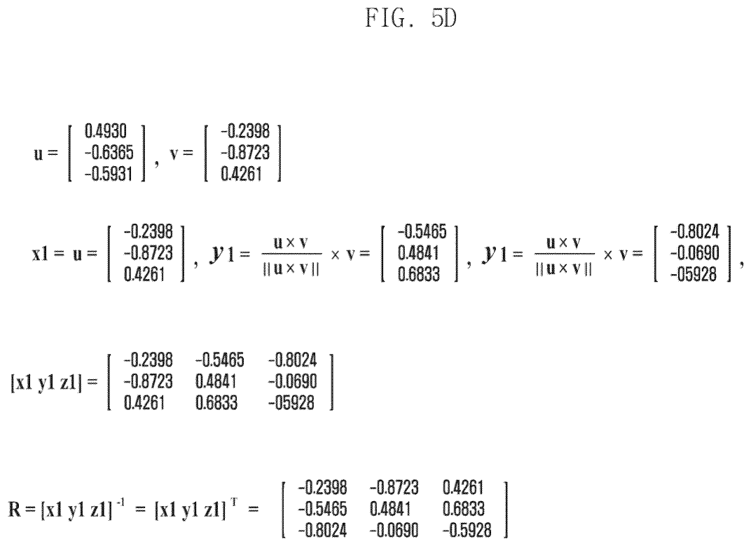

FIGS. 5A, 5B, 5C, and 5D are drawings illustrating an operation for guiding a user to make a specified gesture and calculating a correction value according to an embodiment. This operation may be performed by a ring-type device 500 (e.g., the first electronic device 301).

According to a request of the ring-type device 500, the user may perform a specified gesture (hereinafter, a first gesture). For example, the ring-type device 500, based on data received from a sensor (e.g., a pressure sensor or a biometric sensor) provided in the ring-type device 500, may recognize that the ring-type device 500 is worn on a finger. Based on the recognition of wearing, the ring-type device 500 may request the user to perform a first gesture through an output device. For example, the output device may include a display (e.g., the display 201 in FIG. 2A) provided in the ring-type device 500. The ring-type device 500 may display visual information (e.g., red lights blinking) specified for the first gesture on the display. The user may recognize that correction is required from displayed information and may perform the first gesture. As another example, the output device may include an external electronic device (e.g., the second electronic device 302 in FIG. 3). The ring-type device 500 may transmit a request message to the external electronic device. Based on the reception of the request message, the external electronic device may display visual information (e.g., FIGS. 5A and 5B) indicating the first gesture on the display.

Referring to FIGS. 5A and 5B, the external electronic device, based on the reception of the request message from the ring-type device 500, may display the first image (“510” in FIG. 5A) indicating the gesture of pressing a finger (e.g., index finger) wearing the ring-type device 500 against the thumb, and a second image (“520” in FIG. 5B) indicating the gesture of spreading the index finger from the thumb. The external electronic device may further display a video indicating the gesture of pressing the finger wearing the ring-type device 500 against the thumb and then spreading it from the thumb.

When the user performs the gesture of pressing the finger wearing the ring-type device 500 against the thumb and then spreading it from the thumb, as in the first image 510 and the second image 520, the states of the ring-type device 500 may be those illustrated in FIGS. 5A and 5B.

Assuming that FIG. 5B corresponds to the state before operation, when two fingers are pressed together, the ring-type device 500 tilts along the direction in which the finger wearing the ring-type device 500 moves as shown in FIG. 5A. After that, when the wearing finger is separated from the thumb, the ring-type device 500 returns to the state in FIG. 5B. The vector, indicating acceleration (gravity acceleration) in the direction of gravity detected by the motion sensor (e.g., a three-axis acceleration sensor) in the ring-type device 500 according to the user's operation, may be a first vector u and a second vector v. That is, the ring-type device 500 may obtain the first vector u and the second vector v using raw data generated by the motion sensor. FIG. 5C shows the difference between the first vector u and the second vector v.

Referring to FIG. 5C, it is assumed that both vectors u and v are vectors representing the direction of gravity with a size of 1. Here, if the cross product u×v of u and v is calculated, a single plane where both the vectors u and v exist may be obtained. The ring-type device 500 may obtain a rotation direction from the cross product u×v when the user wears the ring-type device 500. For example, when

$$\frac{u \times v}{‖ u \times v ‖}$$

is defined as an acceleration value z1 in the z-axis direction, the vectors u and v exist on the x-y plane. Based on the state shown in FIG. 5A, the ring-type device 500 may define the vector v as an acceleration value x1 in the x-axis direction (or an acceleration value y1 in the y-axis direction). When assuming as described above, the y-axis acceleration value y1 may be defined as

$$\frac{u \times v}{‖ u \times v ‖} \times v$$

because it is perpendicular to both the x-axis and the z-axis. That is, the ring-type device 500 may obtain the three-axis unit vectors x1, y1, and z1 using Equation 1 below. In Equation 1, ∥u×v∥ indicates the absolute value of u×v.

$$x 1 = v , y 1 = \frac{u \times v}{‖ u \times v ‖} \times v , z 1 = \frac{u \times v}{‖ u \times v ‖} \qquad [ Equation 1 ]$$

Equation 1 is only an example to help understanding, and the embodiments of the disclosure may not be limited thereto. For example, Equation 1 may be modified, applied, or expanded in various ways.

The ring-type device 500 may obtain a radius of rotation (roll) based on the x-axis, a radius of rotation (pitch) based on the y-axis, and a radius of rotation (yaw) based on the z-axis using Equation 1. The ring-type device 500 may obtain a correction value (e.g., a rotation transformation matrix) to correct the acceleration values x1, y1, and z1 into acceleration values x2, y2, and z2 measured when the ring-type device 500 is in a specified wearing state (e.g., the first state in FIG. 4B).

According to an embodiment, the ring-type device 500 may obtain a 3D rotation transformation matrix R that corrects the matrix [x1 y1 z1] into an identity matrix I (or a unit matrix) as shown Equation 2 below. The inverse matrix of [x1 y1 z1] is the rotation transformation matrix R, and R may be obtained using Equation 3 below. Here, since the matrix [x1 y1 z1] is an orthonormal matrix in which each column has a size of 1 and the respective columns are orthogonal to each other, “[x1 y1 z1]−1=[x1 y1 z1]T” is established. In other words, the inverse matrix may be obtained without complex operations by obtaining a transpose matrix that only changes the positions of matrix elements. To help understanding, an example of the process of obtaining the rotation transformation matrix R is illustrated in FIG. 5D.

$$R [ x 1 y 1 z 1 ] = I ( e . g . , I = [ \begin{matrix}1 & 0 & 0 \\ 0 & 1 & 0 \\ 0 & 0 & 1\end{matrix} ] ) \qquad [ Equation 2 ]$$

∴R=[x1y1z1]−1=[x1y1z1]^{T} [Equation 3]

Equations 2 and 3 are only examples to help understanding, and the embodiments of the disclosure may not be limited thereto. For example, Equations 2 and 3 may be modified, applied, or expanded in various ways.

The ring-type device 500 may obtain a first acceleration value (e.g., x-axis acceleration value, y-axis acceleration value, and z-axis acceleration value) using raw data generated by a motion sensor, and correct the first acceleration value using the correction value (rotation transformation matrix R) obtained through the above-described process, thereby obtaining a second acceleration value. Accordingly, a relatively constant second acceleration value may be obtained for the direction of gravity acceleration, regardless of the wearing state of the ring-type device 500. The ring-type device 500 may analyze the movement pattern using the second acceleration value and generate an event corresponding to the movement pattern.

If the angle between the two vectors (e.g., u and v described above) is too small, the correction of the acceleration value may be inaccurate. The wearable electronic device (e.g., the first electronic device 301 in FIG. 3) according to an embodiment may request the user to perform the first gesture again through the output device if the angle between two vectors obtained using the motion sensor is less than or equal to a specified threshold angle value.

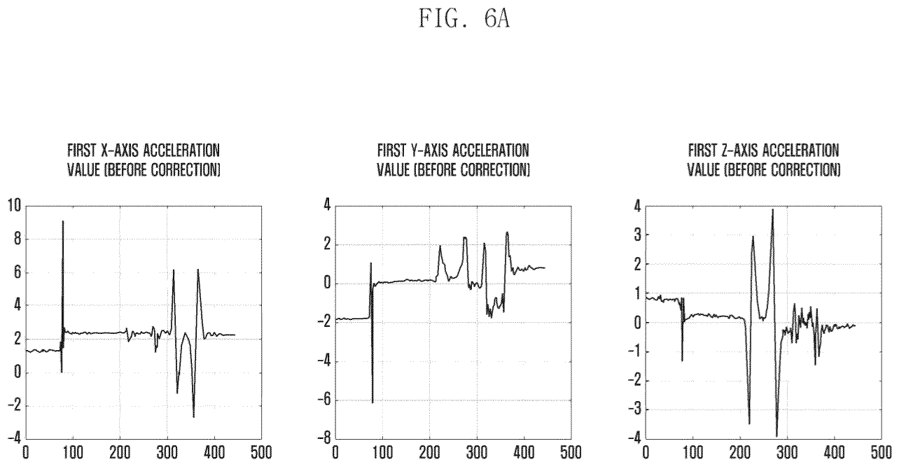

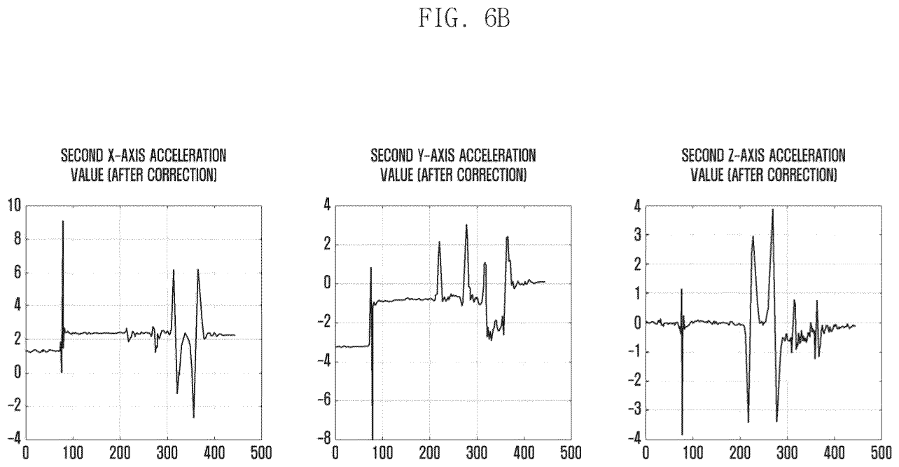

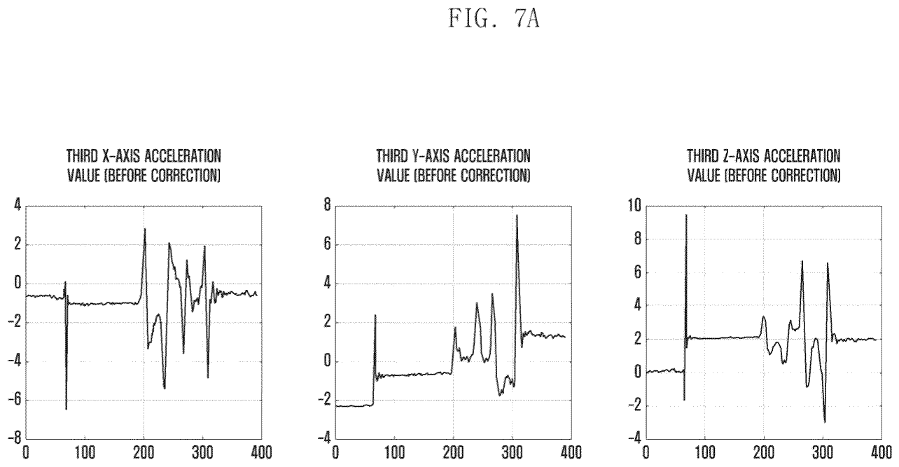

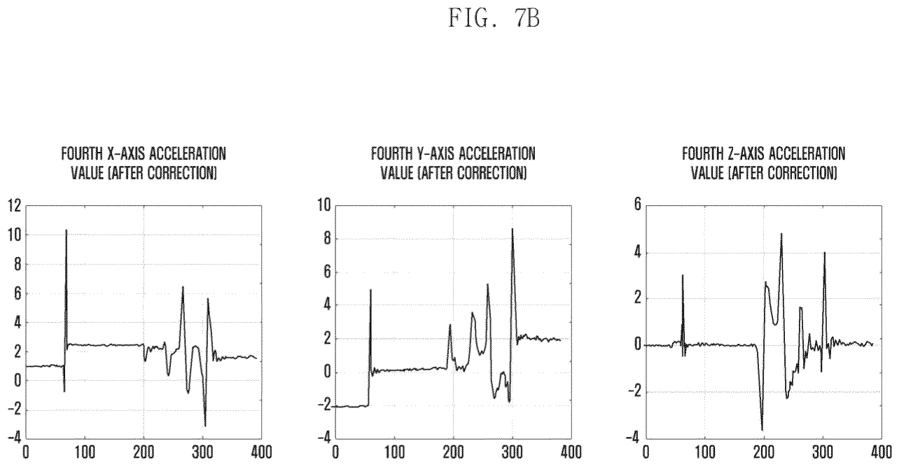

FIGS. 6A and 6B and FIGS. 7A and 7B illustrate cases in which the acceleration value is corrected using the embodiment described with reference to FIGS. 5A, 5B, and 5C above. In this case, the specified gesture requested of the user is assumed to be a gesture in which the user stretches the hand, wearing the ring-type device, straight forward while sitting or standing, and then moves it in the order of right, left, up, and down. In the drawings, the vertical axis represents the size of the acceleration value, and the horizontal axis represents time. For example, the unit of the acceleration value may be m/s2, and the unit of time may be millisecond.

FIGS. 6A and 7A illustrate acceleration values before correction. The user may perform a specified gesture, and accordingly, the ring-type device may obtain a first x-axis acceleration value in the x-axis direction, a first y-axis acceleration value in the y-axis direction, and a first z-axis acceleration value in the z-axis direction, as illustrated in FIG. 6A, using data generated by the motion sensor. The user may change the wearing state (e.g., rotate the ring-type device worn on the finger 90 degrees clockwise) and perform the same gesture, and accordingly, the ring-type device may obtain a third x-axis acceleration value in the x-axis direction, a third y-axis acceleration value in the y-axis direction, and a third z-axis acceleration value in the z-axis direction, as shown in FIG. 7A, using data generated by the motion sensor. Comparing FIG. 6A with FIG. 7A, it may be confirmed that the pattern of acceleration change differs depending on the wearing state.

FIG. 6B and FIG. 7B show acceleration values after correction. The ring-type device may correct the first x-axis acceleration value, the first y-axis acceleration value, and the first z-axis acceleration value into a second x-axis acceleration value, a second y-axis acceleration value, and a second z-axis acceleration value, respectively, using a correction value (rotation transformation matrix R) obtained through the above-described process. The ring-type device may correct the third x-axis acceleration value, the third y-axis acceleration value, and the third z-axis acceleration value into a fourth x-axis acceleration value, a fourth y-axis acceleration value, and a fourth z-axis acceleration value, respectively, using the correction value (rotation transformation matrix R). Comparing FIG. 6B with FIG. 7B, it may be confirmed that the patterns of acceleration change after correction are similar, regardless of the wearing state.

The method of obtaining the correction value described above with reference to FIGS. 5A, 5B, 5C, and 5D is a technique of obtaining the same by recognizing the change in the direction of gravity. Therefore, in a case where the user is identified to be lying down, a method of recognizing a change in linear acceleration and calculating a correction value, based on the recognition, may be more useful than the above method. According to an embodiment, in a case where the user is identified to be lying down, a change in linear acceleration may be recognized, and a correction value may be calculated based on this recognition.

According to the Nyquist sampling theory, if a signal is sampled at regular intervals at a frequency that is twice the highest frequency included in the signal, the signal may be restored to its original signal. Accordingly, if the sampling frequency of a motion sensor (e.g., an acceleration sensor) is less than a certain frequency (e.g., several hundred Hz), a large and slow user gesture (e.g., a gesture of slowly shaking a hand) (hereinafter, referred to as a second gesture) may be required to accurately detect the change in acceleration. If the sampling frequency is equal to or more than a certain frequency, although power consumption of the acceleration sensor relatively increases, the change in acceleration may be accurately detected from a relatively small and fast first gesture (e.g., a gesture of shaking a finger as shown in FIGS. 5A and 5B). According to an embodiment, if the sampling frequency of the acceleration sensor is configured to less than a specified frequency threshold value, a change in linear acceleration may be recognized, and a correction value may be calculated based on the recognition.

An embodiment proposes a method of producing a correction value, based on the direction in which the ring-type device moves, using a second gesture (e.g., pointing a finger wearing a ring-type device forward and shaking the arm up and down). Compared to the first gesture, the second gesture may have a movement direction that is generally the same as the movement direction of the first gesture, but may have a larger radius of rotation and slower movement. Therefore, as described above, in a case where the user is lying down or where the sampling frequency of the acceleration sensor is set to be lower than a certain frequency, the second gesture may be designated as a gesture requested of the user. If the movement direction in the second gesture is configured to be a left-right direction, instead of an up-down direction, the x-axis and z-axis may be swapped in the movement correction procedure to be described below. As described above, the ring-type device may request the user to perform the first gesture or the second gesture, based on at least one of the conditions of the user's state, the sampling frequency (or sampling period), the user's convenience, and the battery capacity of the ring-type device.

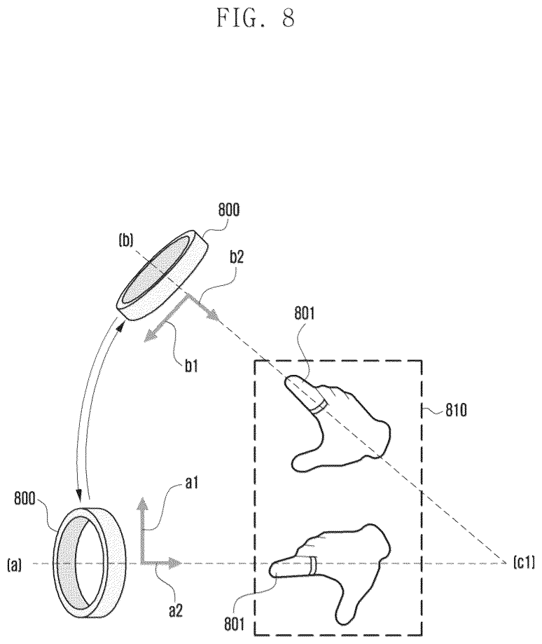

FIG. 8 is a drawing illustrating an operation for calculating a correction value, based on a change in linear acceleration, according to an embodiment. This operation may be performed by a ring-type device 800 (e.g., the first electronic device 301).

According to the request of the ring-type device 800, a user may perform a second gesture 810. For example, the ring-type device 800 may recognize that the ring-type device 800 is worn on a finger, based on data received from a sensor (e.g., a pressure sensor or a biometric sensor) provided in the ring-type device 800. Based on the recognition of wearing, the ring-type device 800 may request the user to perform a second gesture through an output device. For example, the output device may include a display (e.g., the display 201 in FIG. 2A) provided in the ring-type device 800. The ring-type device 800 may display visual information specified for the second gesture on the display. The user may recognize that correction is necessary from the displayed information, and perform the second gesture. As another example, the output device may include an external electronic device (e.g., the second electronic device 302 in FIG. 3). The ring-type device 800 may transmit a request message to the external electronic device. The external electronic device, based on the reception of the request message, may display visual information indicating the second gesture (e.g., an image indicating the second gesture 810) on the display.

Referring to FIG. 8, the external electronic device may display a guide image (or video) indicating the second gesture 810 for the ring-type device 800, based on the reception of the request message from the ring-type device 800. The user may view the guide image, point the finger wearing the ring-type device 800 forward, and then raise the finger upward in the direction opposite the direction of gravity from the position (a). The ring-type device 800 may identify that the finger on which the ring-type device 800 is worn moves upward in the direction opposite the direction of gravity, based on raw data generated by the acceleration sensor. The ring-type device 800 may identify acceleration a from the raw data generated by the acceleration sensor at the time at which the finger starts to move upward. Acceleration a may include acceleration a1 in the tangential direction at the position (a) and centripetal force a2 directed toward c1, which is the center of circular motion, at the position (a). The user may view the guide image, stop the movement at the position (b), and then lower the finger back downward (in the direction of gravity). The ring-type device 800 may detect that a finger wearing the ring-type device 800 moves in the direction of gravity, based on raw data generated by an acceleration sensor. At the time at which the finger starts to move downward (in the direction of gravity), the ring-type device 800 may identify acceleration b from raw data generated by the acceleration sensor. Acceleration b may include acceleration b1 in the tangential direction at the position (b) and centripetal force b2 directed toward c1, which is the center of circular motion, at the position (b).

The ring-type device 800 may define the direction in which the finger wearing the ring-type device 800 is directed as the y-axis. In the circular motion between the positions (a) and (b), the tangential acceleration occurs on a plane perpendicular to the y-axis, that is, on the x-z plane. The ring-type device 800 may define acceleration a1 in the direction from (a) to (b) as x1 on the x-z plane. The ring-type device 800 may define acceleration b1 in the direction from (b) to (a) as the opposite direction of x1, that is, −x1, on the x-z plane. x1 may be defined as Equation 4 below using acceleration a1 and b1.

$$x 1 = \frac{a 1}{‖ a 1 ‖} = - \frac{b 1}{‖ b 1 ‖} \qquad [ Equation 4 ]$$

Equation 4 described above is only an example to help understanding, and the embodiments of the disclosure may not be limited thereto. For example, Equation 4 may be modified, applied, or expanded in various ways.

The y-axis component value is “0” in acceleration a and b. In order to increase the reliability of x1, the ring-type device 800 may also determine x1 using Equation 5 below.

$$x 1 = ( \frac{a 1}{‖ a 1 ‖} - \frac{b 1}{‖ b 1 ‖} ) / 2 \qquad [ Equation 5 ]$$

Equation 5 is only an example to help understanding, and the embodiments of the disclosure may not be limited thereto. For example, Equation 5 may be modified, applied, or expanded in various ways.

Depending on the state in which the ring-type device 800 is worn, the y-axis direction (e.g., the y-axis in FIG. 4A) of the acceleration sensor may match the direction in which the finger 801 is directed or may be exactly opposite thereto. In a state where the y-axis direction substantially matches the direction in which the finger is directed, when the user raises the finger 801 upward at the position (a), the centripetal force acts in the direction (− direction) exactly opposite the direction in which the finger 801 is directed, and when the finger stops at the position (b), the centripetal force acts in the direction (+ direction) in which the finger 801 is directed. When the finger 801 moves back to the position (a), the centripetal force acts in the exact opposite direction (− direction). That is, in a case (hereinafter, Case 1) where the ring-type device 800 is worn on the finger 801 so that the y-axis direction of the acceleration sensor substantially matches the direction in which the finger 801 is directed, the centripetal force is measured in the order of (−)→(+)→(−). In a case (hereinafter, Case 2) where the ring-type device 800 is worn on the finger 801 so that the y-axis direction of the acceleration sensor is exactly opposite to the direction in which the finger 801 is facing, the centripetal force is measured in the order of (+)→(−)→(+). As shown in Equation 6 below, the ring-type device 800 may configure y1 as [0 1 0]T in Case 1, and configure y1 as [0 −1 0]T in Case 2.

$$y 1 = \{ \begin{matrix}{[ \begin{matrix}0 & 1 & 0\end{matrix} ]}^{T} & case 1 \\ {[ \begin{matrix}0 & - 1 & 0\end{matrix} ]}^{T} & case 2\end{matrix} \qquad [ Equation 6 ]$$

Equation 6 described above is only an example to help understanding, and the embodiments of the disclosure may not be limited thereto. For example, Equation 6 may be modified, applied, or expanded in various ways.

Since the z-axis is perpendicular to both the x-axis and the y-axis, the ring-type device 800 may determine z1 to be the cross product x1×y1 of x1 and y1. The ring-type device 800 may obtain a rotation transformation matrix R using Equations 2 and 3 described above.

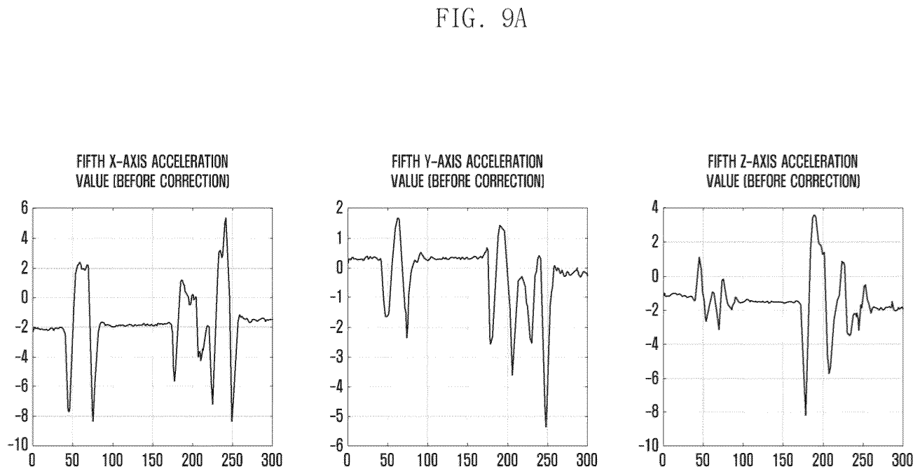

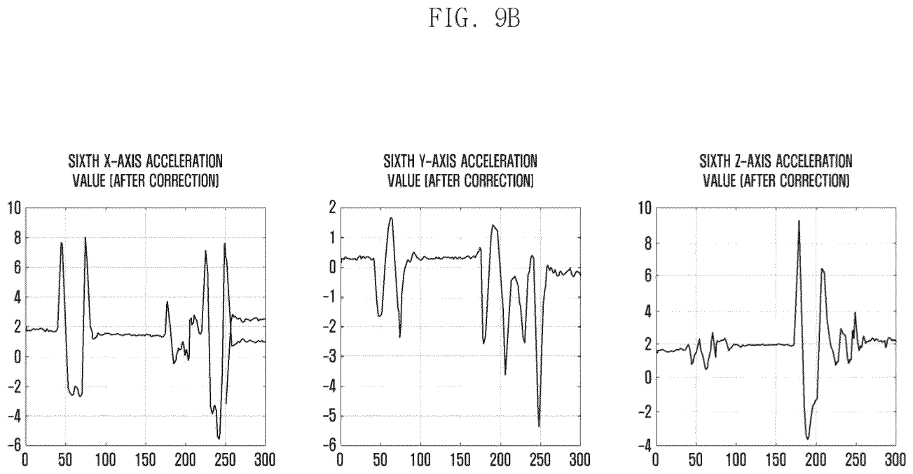

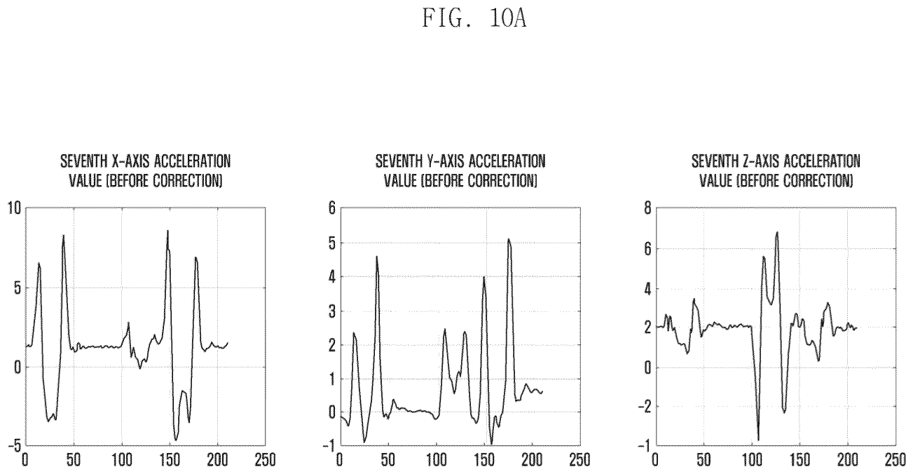

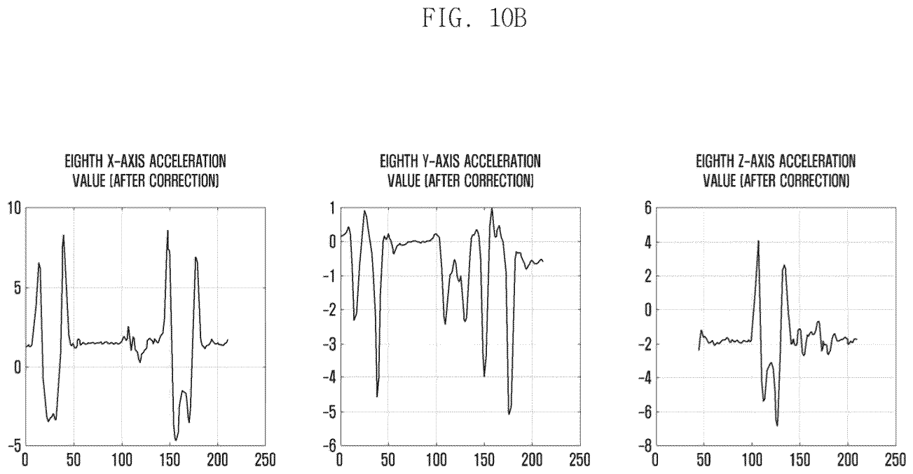

FIGS. 9A and 9B and FIGS. 10A and 10B illustrate cases in which the acceleration values are corrected using the embodiment described with reference to FIG. 8 above. In these cases, the specified gesture requested of the user is assumed to be that the user stretches the hand wearing the ring-type device straight forward, shakes it up and down, and then moves it in the order of right, left, up, and down. In the drawings, the vertical axis represents the size of the acceleration value, and the horizontal axis represents time. For example, the unit of the acceleration value may be m/s2, and the unit of time may be millisecond.

FIGS. 9A and 10A show acceleration values before correction. The user may perform a specified gesture, and accordingly, the ring-type device may obtain a fifth x-axis acceleration value in the x-axis direction, a fifth y-axis acceleration value in the y-axis direction, and a fifth z-axis acceleration value in the z-axis direction using data generated by the motion sensor, as shown in FIG. 9A. The user may change the wearing state (e.g., wear the ring-type device upside down) and perform the same gesture, and accordingly, the ring-type device may obtain a seventh x-axis acceleration value in the x-axis direction, a seventh y-axis acceleration value in the y-axis direction, and a seventh z-axis acceleration value in the z-axis direction, as shown in FIG. 10A, using data generated by the motion sensor. Comparing FIG. 9A with FIG. 10A, it may be confirmed that the pattern of acceleration change differs depending on the wearing state.

FIG. 9B and FIG. 10B show acceleration values after correction. The ring-type device may correct the fifth x-axis acceleration value, the fifth y-axis acceleration value, and the fifth z-axis acceleration value into a sixth x-axis acceleration value, a sixth y-axis acceleration value, and a sixth z-axis acceleration value, respectively, using the correction values (rotation transformation matrix R) obtained through the above-described process. The ring-type device may correct the seventh x-axis acceleration value, the seventh y-axis acceleration value, and the seventh z-axis acceleration value into an eighth x-axis acceleration value, an eighth y-axis acceleration value, and an eighth z-axis acceleration value, respectively, using the correction value (rotation transformation matrix R). Comparing FIG. 9B with FIG. 10B, it may be confirmed that the patterns of acceleration change after correction are similar, regardless of the wearing state.

If the gesture actually performed by the user is different from the specified gesture (e.g., the first gesture or the second gesture described above), the movement pattern may be recognized incorrectly. According to an embodiment, the ring-type device (e.g., 500 or 800), based on the user being guided to perform the first gesture or the second gesture, may obtain information on the characteristics of the acceleration signal from raw data generated by the motion sensor, and compare the characteristic information with preconfigured base information (or reference information). According to an embodiment, the ring-type device may determine whether or not the user performed the specified gesture using an artificial intelligence model (e.g., machine learning classification model). For example, the artificial intelligence model may be stored in the memory (e.g., the first memory 318) of a ring-type device. As another example, the artificial intelligence model may be stored in an external electronic device (e.g., the second electronic device 302). Accordingly, the ring-type device may request a comparison result by transmitting characteristic information to the external electronic device through a communication circuit (e.g., the first communication circuit 311). The ring-type device may receive the comparison result from the external electronic device through a communication circuit. The ring-type device may determine whether or not the user performed the specified gesture, based on the comparison result. If it is determined that the user did not perform the specified gesture, the ring-type device may request the user to perform the preconfigured gesture again. The method of the request is as described above and will be omitted here.

According to an embodiment, the ring-type device (e.g., the first electronic device 301) may monitor the user's state using a motion sensor. For example, the ring-type device may measure the amount of change in acceleration from raw data generated by the motion sensor. The ring-type device may determine the form of the user's gesture, based on the pattern of change in acceleration. For example, the ring-type device may determine whether the pattern of change in acceleration corresponds to the specified gesture (e.g., a gesture of repeatedly shaking an arm when walking or running or a gesture of raising and lowering a wrist to look at a watch). A method of obtaining a correction value, based on the specified gesture being obtained from the raw data, may be provided. According to this method, a user does not need to perform any gesture, which reduces user inconvenience and enhances the ability of the ring-type device to autonomously perform calibration. In various embodiments described herein, this autonomous calibration may supplement, or alternatively replace, the calibration that is performed by guiding the user to perform a gesture.

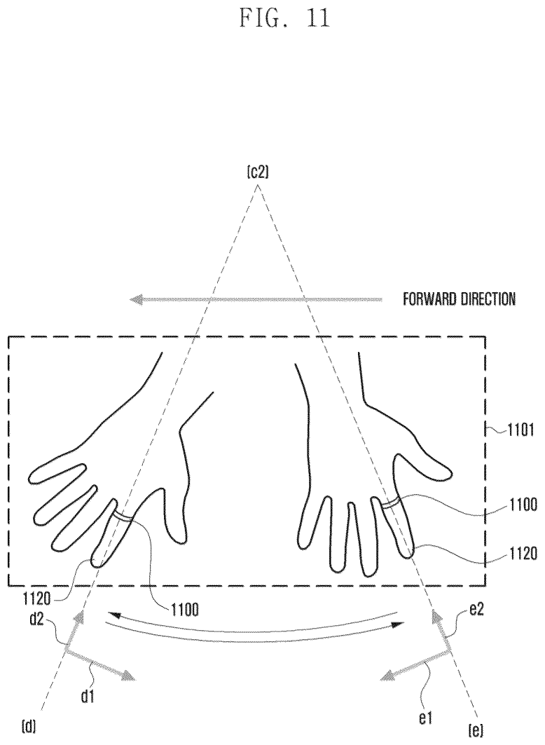

FIG. 11 is a drawing illustrating an operation for calculating a correction value, based on a result of monitoring a user's state, according to an embodiment. This operation may be performed in a ring-type device 1100 (e.g., the first electronic device 301).

According to an embodiment, the ring-type device 1100 (e.g., the first electronic device 301) may detect a vibration pattern (e.g., vibration frequency, amplitude, or intensity) that satisfies a gesture 1101 of shaking an arm when the user walks or runs, from raw data generated by a motion sensor. Based on the gesture 1101 obtained from the raw data, the ring-type device 1100 may determine a forward direction when the user walks or runs, using raw data generated by the motion sensor. The ring-type device 1100 may determine y1 (see FIG. 5C), based on the direction in which the finger 1120 of the user wearing the ring-type device 1100 is directed. For example, the ring-type device may determine the direction of gravity from raw data generated by the motion sensor and produce y1, based on the direction of gravity. While the user is walking or running, the arm moves and accelerations in opposite directions may be continuously detected on the x-z plane. The ring-type device may produce x1 and z1 on the x-z plane using these two accelerations in opposite directions. For example, the ring-type device may calculate the inner product of the two accelerations. The ring-type device 1100 may determine the direction in which walking or running is progressing using raw data generated by the motion sensor. The ring-type device 1100 may determine the wearing state of the ring-type device 1100, based on the calculated inner product and the determined direction (walking or running direction). When the user walks or runs while the ring-type device 1100 is worn on the left hand, the user moves forward in the direction from the little finger to the thumb. The ring-type device may produce z1, based on the forward direction. The ring-type device may determine x1 as the cross product of y1 and z1. The ring-type device may obtain a rotation transformation matrix R using Equation 3 described above.

Referring to FIG. 11, the ring-type device 1100 may identify the direction in which the user moves forward, based on raw data generated by the acceleration sensor. The ring-type device 1100 may identify that the finger wearing the ring-type device 1100 stops and then moves backward in the direction opposite the forward direction, based on raw data generated by the acceleration sensor. According to those illustrated in the drawing, the backward movement of the finger wearing the ring-type device 1100 may start at the position (d). The ring-type device 1100 may identify acceleration d from raw data generated by the acceleration sensor at the time at which the backward movement starts. Acceleration d may include acceleration d1 in the tangential direction at the position (d) and centripetal force d2 directed toward c2, which is the center of the circular motion, at the position (d). The ring-type device 1100 may identify that the finger wearing the ring-type device 1100 stops and then moves forward, based on raw data generated by the acceleration sensor. According to those illustrated in the drawing, the forward movement of the finger wearing the ring-type device 1100 may start at the position (e). The ring-type device 1100 may identify acceleration e from raw data generated by the acceleration sensor at the time at which the forward movement starts. Acceleration e may include acceleration e1 in the tangential direction at the position (e) and centripetal force e2 directed toward c2, which is the center of the circular motion, at the position (e).

The ring-type device 1100 may determine the direction in which the finger wearing the ring-type device 1100 is directed as the y-axis. In the circular motion between the positions (d) and (e), the tangential acceleration occurs on a plane perpendicular to the y-axis, that is, on the x-z plane. The ring-type device 800 may determine acceleration d1 in the direction from (d) to (e) on the x-z plane as x1 (see FIG. 5C). The ring-type device 1100 may determine acceleration e1 in the direction from (e) to (d) on the x-z plane as the opposite direction of x1, i.e., −x1. x1 may be defined as Equations 4 and 5 described above using acceleration d1 and e1. That is, a1 and b1 in Equations 4 and 5 described above may be replaced with d1 and e1, respectively. The ring-type device 1100 may obtain y1 using Equation 6 described above. The ring-type device 1100 may determine z1 as the cross product x1×y1 of x1 and y1 (see FIG. 5C). The ring-type device 800 may obtain a rotation transformation matrix R using Equations 2 and 3 described above.

A watch-type device may be worn on the same hand as the ring-type device. The user performs a gesture of raising and lowering the wrist (hereinafter, a wrist gesture) to view or turn on the screen of the watch-type device. A method of obtaining a correction value may be provided based on the recognition of the wrist gesture. According to an embodiment, the ring-type device may detect a wrist gesture as an action of the user to view the watch from raw data generated by the motion sensor. When the user raises and then lowers the wrist back, the hand rotates around the elbow, so that a centripetal force that pulls the ring-type device toward the inside of the finger is applied. The ring-type device may determine the direction of the centripetal force and calculate y1, based on this. In addition, when raising the hand, acceleration occurs in the direction from the little finger to the thumb, and when lowering the hand, acceleration occurs in the opposite direction from the thumb to the little finger. These two directions of acceleration are opposite to each other. The ring-type device may produce x1 by reversing one direction and taking the average (see Equation 5). Accordingly, the accuracy of calculation for x1 may be further improved. In addition, the ring-type device may further improve the accuracy of calculation for x1 by obtaining acceleration values from repeated wrist gestures and taking an average. The ring-type device 800 may determine z1 as the cross product x1×y1 of x1 and y1. The ring-type device 800 may obtain a rotation transformation matrix R using Equation 3 described above.

In the method of indirectly obtaining the correction value by monitoring the user's state as described above, the ring-type device may accumulate acceleration values measured from gestures that occur repeatedly in daily life, and calculate the correction value using the accumulated statistics. The accuracy of the correction value obtained by the calculation using statistics may be higher than that calculated based on a one-time measurement.

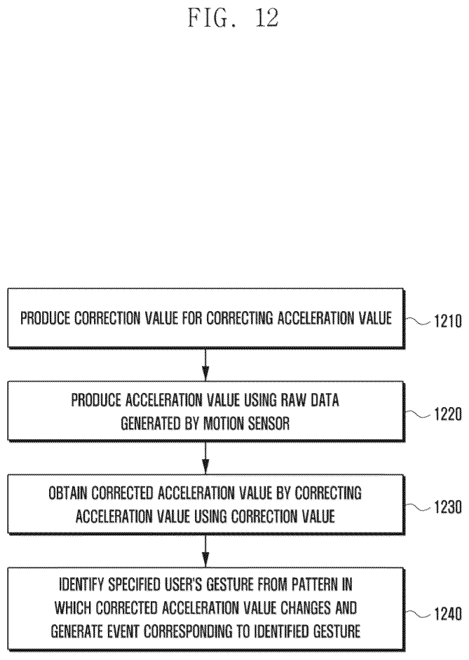

FIG. 12 is a flowchart illustrating an overall operation of a ring-type device (e.g., the first electronic device 301) to generate an event in response to a user's gesture according to an embodiment. According to an embodiment, a processor (e.g., the first processor 319) in the ring-type device may be configured to perform the operation in FIG. 12. According to an embodiment, instructions stored in a memory (e.g., the first memory 318 and/or the internal memory of the first processor 319) may cause, when executed by the processor, the electronic device to perform the operation in FIG. 12. In describing the operation in FIG. 12, the description that has already been given above will be omitted or briefly mentioned.

In the following embodiment, respective operations may be performed sequentially, but are not necessarily performed sequentially. For example, the operations may be performed in a different sequence, and at least two operations may be performed in parallel.

According to an embodiment, in operation 1210, the ring-type device may produce a correction value for correcting the acceleration value calculated using data generated by a motion sensor (e.g., the motion sensor 312 in FIG. 3). This correction value may be a value (e.g., the rotation transformation matrix R described above) for obtaining substantially the same acceleration value when the user makes substantially the same gesture, regardless of the direction or state in which the ring-type device is worn on the user's finger.

According to an embodiment, in operation 1220, the ring-type device may produce an acceleration value using raw data generated by the motion sensor. For example, the ring-type device may obtain an acceleration value in the x-axis direction, an acceleration value in the y-axis direction, and an acceleration value in the z-axis direction from the raw data of the motion sensor. For example, the acceleration values illustrated in FIGS. 6A, 7A, 9A, and 10A may correspond to the acceleration values obtained in operation 1220.

According to an embodiment, in operation 1230, the ring-type device may obtain a corrected acceleration value by correcting the acceleration value using the correction value. For example, the acceleration values illustrated in FIGS. 6B, 7B, 9B, and 10B may correspond to the acceleration values obtained through correction in operation 1230.

According to an embodiment, in operation 1240, the ring-type device may identify a specified user's gesture from the pattern in which the corrected acceleration value changes, and generate an event corresponding to the identified gesture. For example, the ring-type device may transmit the event to an external electronic device (e.g., the second electronic device 302) through a communication circuit (e.g., the first communication circuit 311). In response to the reception of the event, the external electronic device may perform a function specified for the event (e.g., answering a call, ending a call, playing media, pausing playback, taking a picture, adjusting the volume, or changing TV channels).

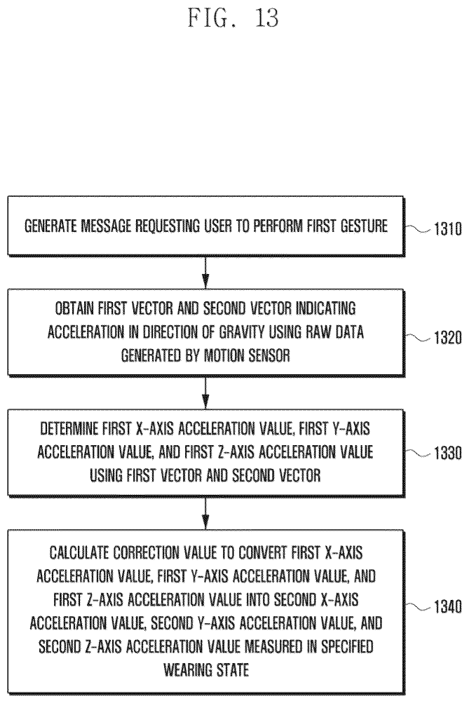

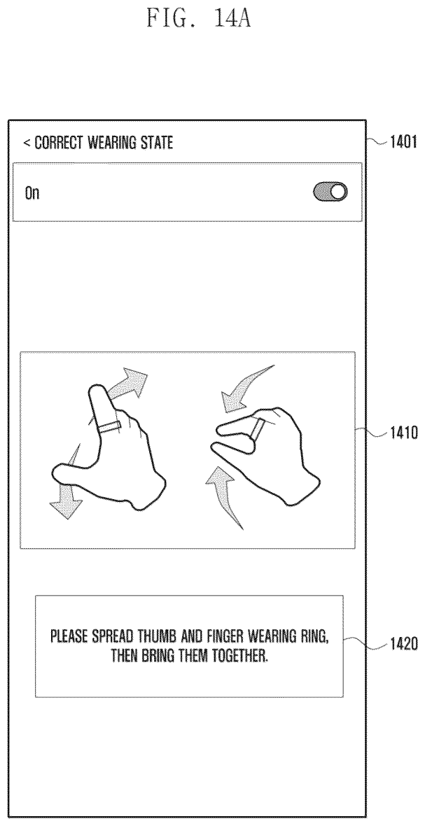

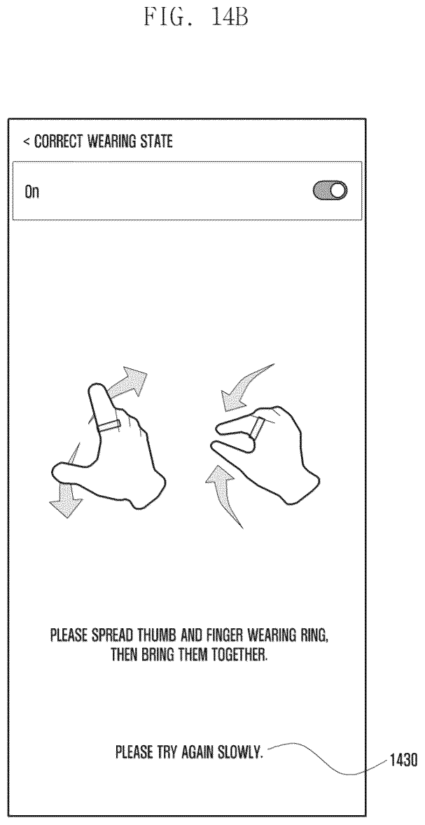

FIG. 13 is a flowchart illustrating an operation (e.g., operation 1210 in FIG. 12) of a ring-type device (e.g., the first electronic device 301) to obtain a correction value according to an embodiment. In describing the operation in FIG. 13, the description that has already been given above will be omitted or briefly mentioned. FIGS. 14A and 14B illustrate examples of user interface (UI) screens that may be presented to a user regarding a gesture that is to be performed by the user.

According to an embodiment, a processor (e.g., the first processor 319) in the ring-type device may be configured to perform the operation in FIG. 13. According to an embodiment, instructions stored in a memory (e.g., the first memory 318 and/or the internal memory of the first processor 319) may cause, when executed by the processor, the electronic device to perform the operation in FIG. 13. According to an embodiment, the ring-type device may recognize that the ring-type device 500 is worn on a finger, based on data received from a sensor (e.g., a pressure sensor or a biometric sensor) provided in the ring-type device. Based on the recognition of wearing, the operation in FIG. 13 may be initiated.

In the following embodiment, respective operations may be performed sequentially, but are not necessarily performed sequentially. For example, the operations may be performed in a different sequence, and at least two operations may be performed in parallel.

According to an embodiment, in operation 1310, the ring-type device may generate a message requesting the user to perform a first gesture. For example, the ring-type device may generate a message requesting the user to perform a first gesture and transmit it to an external electronic device (e.g., the second electronic device 302) through a communication circuit (e.g., the first communication circuit 311). In response to the reception of the request message, the external electronic device may display a first UI screen 1401 on the display. Referring to FIG. 14A, the external electronic device may include an image 1410 indicating a gesture that is to be performed by the user and a description 1420 showing how to perform the corresponding gesture in the first UI screen 1401 and then display it on the display. According to an embodiment, the ring-type device may obtain information about the characteristics of an acceleration signal from raw data generated by a motion sensor, and compare the characteristic information with pre-stored reference information. If they do not match each other as a result of the comparison, the ring-type device may transmit a message requesting the user to perform the first gesture again. Referring to FIG. 14B, in response to the reception of the re-request message, the external electronic device may include a phrase 1430 indicating the re-request in the first UI screen 1401, and then display it on the display.

After requesting the user to perform the first gesture, in operation 1320, the ring-type device may obtain a first vector and a second vector indicating acceleration in the direction of gravity using raw data generated by the motion sensor. For example, “u” and “v” expressed in Equation 1 above may correspond to the first vector and the second vector obtained in operation 1320.

According to an embodiment, in operation 1330, the ring-type device may determine a first x-axis acceleration value in the x-axis direction, a first y-axis acceleration value in the y-axis direction, and a first z-axis acceleration value in the z-axis direction using the first vector and the second vector. For example, x1, y1, and z1 expressed in Equation 1 above may correspond to the first x-axis acceleration value, the first y-axis acceleration value, and the first z-axis acceleration value.

According to an embodiment, in operation 1340, the ring-type device may calculate a correction value that converts the first x-axis acceleration value, the first y-axis acceleration value, and the first z-axis acceleration value into a second x-axis acceleration value, a second y-axis acceleration value, and a second z-axis acceleration value measured when the ring-type device is in a specified wearing state (e.g., the first state in FIG. 4B). For example, the ring-type device may calculate a rotation transformation matrix R corresponding to the correction value using Equation 3.

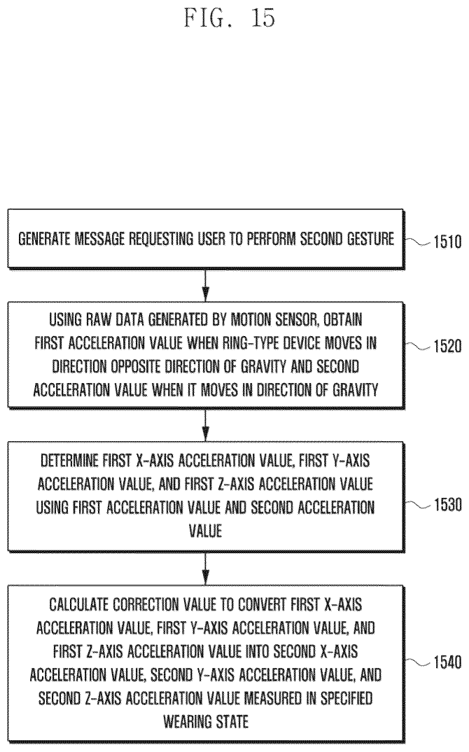

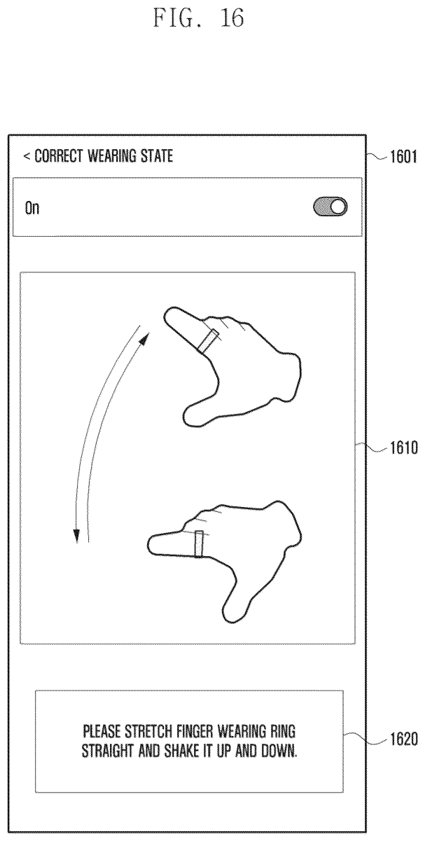

FIG. 15 is a flowchart illustrating the operation (e.g., operation 1210 in FIG. 12) of a ring-type device (e.g., the first electronic device 301) for obtaining a correction value according to an embodiment. In describing the operation in FIG. 15, the description that has already been given above will be omitted or briefly mentioned. FIG. 16 illustrates an example of a user interface (UI) screen that may be presented to a user regarding a gesture that is to be performed by the user.

According to an embodiment, a processor (e.g., the first processor 319) in the ring-type device may be configured to perform the operation in FIG. 15. According to an embodiment, instructions stored in a memory (e.g., the first memory 318 and/or the internal memory of the first processor 319) may cause, when executed by the processor, the electronic device to perform the operation in FIG. 15. In describing the operation in FIG. 15, the description that has already been given above will be omitted or briefly mentioned.

The ring-type device may recognize that the ring-type device 500 is worn on a finger, based on data received from a sensor (e.g., a pressure sensor or a biometric sensor) provided in the ring-type device. Based on the recognition of wearing, the ring-type device may perform the operation in FIG. 15.

According to an embodiment, based on the sampling frequency of a motion sensor (e.g., an acceleration sensor) being configured to be less than a specified threshold frequency value (e.g., several hundred Hz), the ring-type device may perform the operation in FIG. 15. Based on the sampling frequency being configured to be equal to or more than the threshold frequency value, the ring-type device may perform the operation in FIG. 13 described above.

According to an embodiment, the ring-type device may measure the amount of change in acceleration from raw data generated by the motion sensor, and determine the user's state, based on the pattern of change in acceleration. For example, the ring-type device may determine that the pattern of change in acceleration corresponds to a gesture performed by the user while lying down. When the user is lying down, the ring-type device may perform the operation in FIG. 15. When the pattern of change in acceleration is determined to correspond to a gesture performed by the user while sitting or standing, the operation in FIG. 13 described above may be performed.

In the following embodiment, respective operations may be performed sequentially, but are not necessarily performed sequentially. For example, the operations may be performed in a different sequence, and at least two operations may be performed in parallel.

According to an embodiment, in operation 1510, the ring-type device may generate a message requesting the user to perform a second gesture (e.g., the second gesture 810). For example, the ring-type device, based on the recognition of wearing, may generate a message requesting the user to perform the second gesture, and transmit the message to an external electronic device (e.g., the second electronic device 302) through a communication circuit (e.g., the first communication circuit 311). In response to the reception of the request message, the external electronic device may display a UI screen 1601 on the display. Referring to FIG. 16, the external electronic device may indicate an image 1610 indicating a gesture that is to be performed by the user and a description 1620 showing how to perform the corresponding gesture in the UI screen 1601, and then display it on the display. According to an embodiment, the ring-type device may obtain information about the characteristics of an acceleration signal from raw data generated by the motion sensor, and compare the characteristic information with pre-stored reference information. If they do not match each other as a result of the comparison, the ring-type device may transmit a message requesting the user to perform the second gesture again. In response to the reception of the re-request message, the external electronic device may include a phrase indicating the re-request in the UI screen 1601, and then display it on the display.

According to an embodiment, in operation 1520, the ring-type device may identify, using raw data generated by the motion sensor, a first acceleration value when the ring-type device moves in the direction opposite the direction of gravity, and obtain a second acceleration value when the ring-type device moves in the direction of gravity. For example, acceleration a and b described above with reference to FIG. 8 may correspond to the first acceleration value and the second acceleration value, respectively.

According to an embodiment, in operation 1530, the ring-type device may determine a first x-axis acceleration value in the x-axis direction, a first y-axis acceleration value in the y-axis direction, and a first z-axis acceleration value in the z-axis direction using the first acceleration value and the second acceleration value. For example, x1 and y1 expressed in Equations 5 and 6 may correspond to the first x-axis acceleration value and the first y-axis acceleration value. The cross product of x1 and y1 may correspond to the first z-axis acceleration value.

According to an embodiment, in operation 1540, the ring-type device may calculate a correction value that converts the first x-axis acceleration value, the first y-axis acceleration value, and the first z-axis acceleration value into a second x-axis acceleration value, a second y-axis acceleration value, and a second z-axis acceleration value measured when the ring-type device is in a specified wearing state (e.g., the first state in FIG. 4B). For example, the ring-type device may calculate a rotation transformation matrix R corresponding to the correction value using Equation 3.

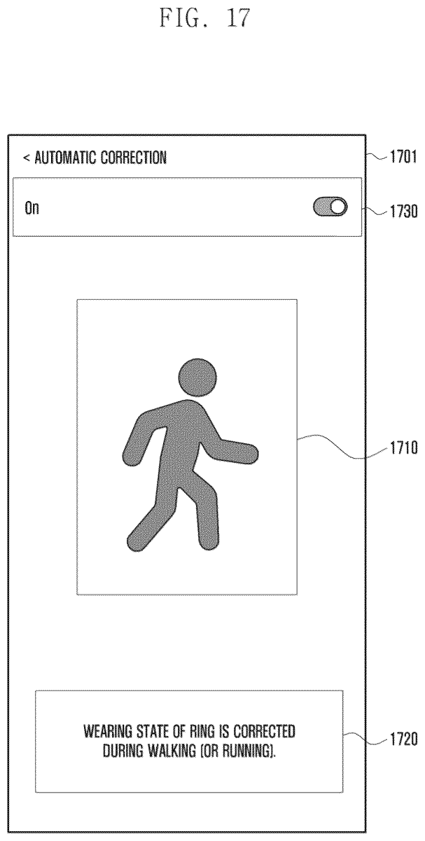

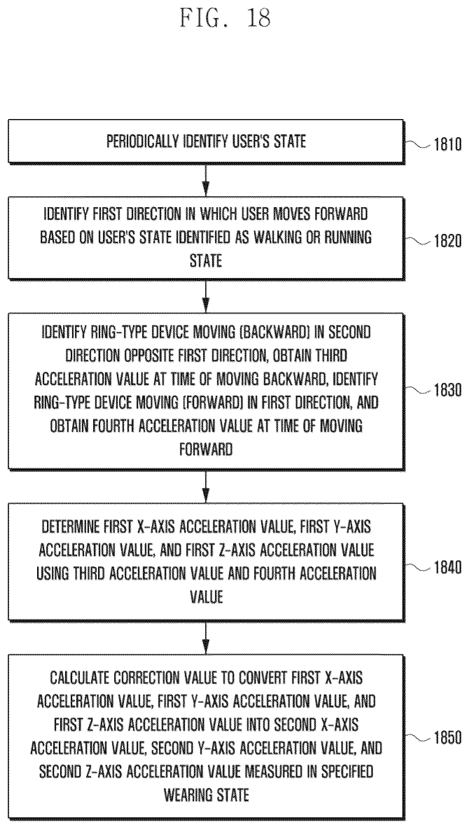

FIG. 17 illustrates an example of a user interface (UI) screen that allows a user to configure whether to automatically perform correction. FIG. 18 is a flowchart illustrating an operation (e.g., the operation 1210 in FIG. 12) of a ring-type device (e.g., the first electronic device 301) for obtaining a correction value according to an embodiment. In describing the operation in FIG. 18, the description that has already been given above will be omitted or briefly mentioned.

Referring to FIG. 17, an external electronic device (e.g., the second electronic device 302) may display a UI screen 1701 on the display. For example, the external electronic device may display a UI screen 1701 including an image 1710 indicating walking (or running), a phrase 1720 indicating execution of automatic correction, and a menu 1730 that allows a user to select whether to activate automatic correction. The external electronic device may transmit information indicating whether automatic correction is activated to the ring-type device. The ring-type device may identify whether automatic correction is activated from the information received through a communication circuit (e.g., the first communication circuit 311). If it is identified that automatic correction is activated, the ring-type device may perform the operation in FIG. 18.

In the following embodiment, respective operations may be performed sequentially, but are not necessarily performed sequentially. For example, the operations may be performed in a different sequence, and at least two operations may be performed in parallel.

According to an embodiment, in operation 1810, the ring-type device may periodically identify the user's state using raw data generated by a motion sensor. According to an embodiment, the ring-type device may measure the amount of change in acceleration at specified intervals from the raw data generated by the motion sensor. The ring-type device may determine the user's state, based on the pattern of change in acceleration. If the pattern of change in acceleration corresponds to a gesture of the user repeatedly shaking the arm, the ring-type device may determine the user's state as a walking or running state.

Based on the user's state identified as a walking or running state as a result of the monitoring, in operation 1820, the ring-type device may determine a first direction in which the user wearing the ring-type device moves forward using raw data generated by the motion sensor.

According to an embodiment, in operation 1830, the ring-type device, based on raw data generated by the motion sensor, may identify that the ring-type device moves (backward) in a second direction opposite the first direction, and obtain a third acceleration value (e.g., acceleration d described above in relation to FIG. 11) at the time of moving backward. In addition, in operation 1830, the ring-type device may identify that the ring-type device moves (forward) in the first direction, based on raw data generated by the motion sensor, and obtain a fourth acceleration value (e.g., acceleration e described above in relation to FIG. 11) at the time of forward movement. According to an embodiment, the ring-type device may periodically perform operation 1830 when the user is walking or running, and accumulate acceleration values obtained by performing this operation. The ring-type device may obtain a third acceleration value and a fourth acceleration value by averaging the accumulated acceleration values.

According to an embodiment, in operation 1840, the ring-type device may determine a first x-axis acceleration value in the x-axis direction, a first y-axis acceleration value in the y-axis direction, and a first z-axis acceleration value in the z-axis direction using the third acceleration value and the fourth acceleration value. For example, x1 and y1 expressed in Equations 5 and 6 may correspond to the first x-axis acceleration value and the first y-axis acceleration value. The cross product of x1 and y1 may correspond to the first z-axis acceleration value.

According to an embodiment, in operation 1850, the ring-type device may calculate a correction value that converts the first x-axis acceleration value, the first y-axis acceleration value, and the first z-axis acceleration value into a second x-axis acceleration value, a second y-axis acceleration value, and a second z-axis acceleration value measured when the ring-type device is in a specified wearing state (e.g., the first state in FIG. 4B). For example, the ring-type device may produce a rotation transformation matrix R corresponding to the correction value using Equation 3.

The correction value calculation method according to the above-described embodiment may be applied to other wearable electronic devices (e.g., watch-type devices, HMD devices, wireless headsets, and wireless earphones), in addition to the ring-type device. In the case of a watch-type device, information related to the wearing state (e.g., the left and right hand to wear the device) may be input into the watch-type device in advance. However, re-entering information every time the wearing position changes may be inconvenient for the user. Therefore, the watch-type device may produce a correction value according to the above-described embodiment and use it to correct the acceleration value, thereby accurately identifying the user's movement or gesture. In the case of wireless earphones (e.g., TWS-true wireless stereo or OWS-open wireless stereo), wireless headsets (e.g., Over-ear/On-ear type), or HMD devices, the wearing state may vary slightly because each person has a different ear shape or head shape. The wearable electronic device may correct individual deviations using the above-described embodiments, thereby improving the accuracy of recognizing the user's movement or gesture.

According to an embodiment, an electronic device (e.g., the first electronic device 301) capable of being worn on a finger includes a motion sensor configured to detect movement of the electronic device, a processor, and a memory configured to store instructions. The instructions, when executed by the processor, may cause the electronic device to generate a first message requesting a user to perform a specified first gesture. The instructions may cause the electronic device to obtain, at least based both on the generation of the first message and data generated by the motion sensor, a first vector and a second vector indicating acceleration in a direction of gravity. The instructions may cause the electronic device to determine a first x-axis acceleration value in an x-axis direction, a first y-axis acceleration value in a y-axis direction, and a first z-axis acceleration value in a z-axis direction using the first vector and the second vector. The instructions may cause the electronic device to calculate (or determine) a correction value of converting the first x-axis acceleration value, the first y-axis acceleration value, and the first z-axis acceleration value into a second x-axis acceleration value, a second y-axis acceleration value, and a second z-axis acceleration value measured when the electronic device is in a specified wearing state. The instructions may cause the electronic device to, based on the correction value, calibrate (or correct) data acquired from the motion sensor.

According to an embodiment, there is provided a recording medium storing instructions readable by a processor of an electronic device (e.g., the first electronic device 301) capable of being worn on a finger. The instructions, when executed by the processor, may cause the electronic device to generate a first message requesting a user to perform a specified first gesture. The instructions may cause the electronic device to obtain, at least based both on the generation of the first message and data generated by a motion sensor of the electronic device, a first vector and a second vector indicating acceleration in the direction of gravity. The instructions may cause the electronic device to determine, based on the first vector and the second vector, a first x-axis acceleration value in an x-axis direction, a first y-axis acceleration value in a y-axis direction, and a first z-axis acceleration value in a z-axis direction. The instructions may cause the electronic device to calculate (or determine) a correction value of converting the first x-axis acceleration value, the first y-axis acceleration value, and the first z-axis acceleration value into a second x-axis acceleration value, a second y-axis acceleration value, and a second z-axis acceleration value measured when the electronic device is in a specified wearing state. The instructions may cause the electronic device to calibrate (or correct), based on the correction value, data acquired from the motion sensor.

The electronic device may include a communication circuit configured to enable the electronic device to wirelessly communicate with an external electronic device (e.g., the second electronic device 302). The instructions may cause the electronic device to transmit the first message to the external electronic device through the communication circuit, and obtain, based on data generated by the motion sensor after the first message is transmitted to the external electronic device, a first vector and a second vector indicating acceleration in a direction of gravity. The data generated by the motion sensor after the first message is transmitted to the external electronic device is at least part of the data generated by the motion sensor.

The instructions may cause the electronic device to retransmit the first message, based on determining that an angle between the first vector and the second vector is less than or equal to a specified threshold angle value.

The instructions may cause the electronic device to generate the first message and obtain the first vector and the second vector, based on a sampling frequency being set to be equal to or more than a specified threshold frequency value in the motion sensor.

The instructions may cause the electronic device to, based on the sampling frequency being set to be less than the specified threshold frequency value, generate a second message requesting the user to perform a specified second gesture, obtain, at least based both on the generation of the second message and data generated by the motion sensor, a first acceleration value when the electronic device moves in a direction opposite the direction of gravity and a second acceleration value when the electronic device moves in the direction of gravity, and determine, based on the first acceleration value and the second acceleration value, the first x-axis acceleration value, the first y-axis acceleration value in the y-axis direction, and the first z-axis acceleration value in the z-axis direction.

The instructions may cause the electronic device to determine a user's state based on data generated by the motion sensor, and, based on the determination of the user's state as being any of a sitting state and a standing state, generate the first message and obtain the first vector and the second vector.

The instructions may cause the electronic device to, based on the determination of the user's state as being a lying down state, generate a second message requesting the user to perform a specified second gesture, obtain, at least based both on the generation of the second message and data generated by the motion sensor, a first acceleration value when the electronic device moves in a direction opposite the direction of gravity and a second acceleration value when the electronic device moves in the direction of gravity, and determine, based on the first acceleration value and the second acceleration value, the first x-axis acceleration value, the first y-axis acceleration value in the y-axis direction, and the first z-axis acceleration value in the z-axis direction.

According to an embodiment, an electronic device (e.g., the first electronic device 301) capable of being worn on a finger may include a communication circuit for the electronic device to wirelessly communicate with an external electronic device, a motion sensor configured to detect movement of the electronic device, a processor, and a memory configured to store instructions, wherein the instructions, when executed by the processor, may cause the electronic device to periodically identify a user's state based on data generated by the motion sensor. The instructions may cause the electronic device to identify, based on the user's state being identified as any of a walking state and a running state and based on data generated by the motion sensor, a first direction in which a user, as wearing the electronic device, moves forward. The instructions may cause the electronic device to identify, based on data generated by the motion sensor, that the electronic device moves backward in a second direction opposite the first direction, and obtain a third acceleration value at a time as which the electronic device is determined to be moving backward. The instructions may cause the electronic device to identify, based on data generated by the motion sensor, that the electronic device moves forward in the first direction, and obtain a fourth acceleration value at a time at which the electronic device is determined to be moving forward. The instructions may cause the electronic device to determine, based on the third acceleration value and the fourth acceleration value, a first x-axis acceleration value in an x-axis direction, a first y-axis acceleration value in a y-axis direction, and a first z-axis acceleration value in a z-axis direction. The instructions may cause the electronic device to calculate (or determine) a correction value to convert the first x-axis acceleration value, the first y-axis acceleration value, and the first z-axis acceleration value into a second x-axis acceleration value, a second y-axis acceleration value, and a second z-axis acceleration value measured when the electronic device is in a specified wearing state. The instructions may cause the electronic device to calibrate (or correct), based on the correction value, data acquired from the motion sensor.

According to an embodiment, there is provided a method for operating an electronic device (e.g., the first electronic device 301) capable of being worn on a finger. The method may include generating a first message requesting a user to perform a specified first gesture. The method may include obtaining, at least based both on the generation of the first message and data generated by a motion sensor of the electronic device, a first vector and a second vector indicating acceleration in a direction of gravity. The method may include determining a first x-axis acceleration value in an x-axis direction, a first y-axis acceleration value in a y-axis direction, and a first z-axis acceleration value in a z-axis direction using the first vector and the second vector. The method may include calculating (or determining) a correction value of converting the first x-axis acceleration value, the first y-axis acceleration value, and the first z-axis acceleration value into a second x-axis acceleration value, a second y-axis acceleration value, and a second z-axis acceleration value measured when the electronic device is in a specified wearing state. The method may include calibrating (or correcting), based on the correction value, data acquired from the motion sensor.

The electronic device may include a communication circuit configured to enable the electronic device to wireless communication with an external electronic device (e.g., the second electronic device 302). The generating may include transmitting the first message to the external electronic device through the communication circuit. The obtaining may include obtaining a first vector and a second vector indicating acceleration in the direction of gravity based on data generated by the motion sensor after the first message is transmitted. The data generated by the motion sensor after the first message is transmitted to the external electronic device is at least part of the data generated by the motion sensor.

The method may further include retransmitting the first message, based on determining that an angle between the first vector and the second vector is less than or equal to a specified threshold angle value.

The generating and the obtaining may be performed based on a sampling frequency being configured to be equal to or more than a specified threshold frequency value in the motion sensor.

The method may further include generating a second message requesting the user to perform a specified second gesture, based on the sampling frequency being configured to be less than the threshold frequency value, obtaining, at least based both on the generation of the second message and data generated by the motion sensor, a first acceleration value when the electronic device moves in a direction opposite the direction of gravity and a second acceleration value when the electronic device moves in the direction of gravity, and determining, based on the first acceleration value and the second acceleration value, the first x-axis acceleration value, the first y-axis acceleration value in the y-axis direction, and the first z-axis acceleration value in the z-axis direction.

The method may further include determining a user's state based on data generated by the motion sensor. The generating and the obtaining may be performed based on determining the user's state as being any of a sitting state and a standing state.

Based on the determining the user's state as being a lying down state, the method may further include generating a second message requesting the user to perform a specified second gesture, obtaining, at least based both on the generation of the second message and data generated by the motion sensor, a first acceleration value when the electronic device moves in a direction opposite the direction of gravity and a second acceleration value when the electronic device moves in the direction of gravity, and determining, based on the first acceleration value and the second acceleration value, the first x-axis acceleration value, the first y-axis acceleration value in the y-axis direction, and the first z-axis acceleration value in the z-axis direction.

In the above description, the terms “first,” “second,” and “third” are only used to distinguish components with the same name, and do not have any special meaning, such as importance or order.

In this disclosure, the expression “connection” may not only indicate a direct connection between components, but may also indicate an electrical connection between components with another component (e.g., a resistor, an inductor, etc.) interposed therebetween.

The electronic device according to various embodiments set forth herein may be one of various types of electronic devices. The electronic device may include, for example, a portable communication device (e.g., a smart phone), a computer device, a portable multimedia device, a portable medical device, a camera, a wearable device, or a home appliance. The electronic device according to embodiments of the disclosure is not limited to those described above.

It should be appreciated that the embodiments and the terms used therein are not intended to limit the technological features set forth herein to particular embodiments and the disclosure includes various changes, equivalents, or alternatives for a corresponding embodiment. With regard to the description of the drawings, similar reference numerals may be used to designate similar or relevant elements. A singular form of a noun corresponding to an item may include one or more of the items, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one or all possible combinations of the items enumerated together in a corresponding one of the phrases. Such terms as “a first,” “a second,” “the first,” and “the second” may be used to simply distinguish a corresponding element from another, and does not limit the elements in other aspect (e.g., importance or order). If an element (e.g., a first element) is referred to, with or without the term “operatively” or “communicatively”, as “coupled with/to” or “connected with/to” another element (e.g., a second element), it means that the element may be coupled/connected with/to the other element directly (e.g., wiredly), wirelessly, or via a third element.

As used in various embodiments of the disclosure, the term “module” may include a unit implemented in hardware, software, or firmware, and may be interchangeably used with other terms, for example, “logic,” “logic block,” “component,” or “circuit”. The “module” may be a single integrated component, or a minimum unit or part thereof, adapted to perform one or more functions. According to an embodiment, the “module” may be implemented in the form of an application-specific integrated circuit (ASIC).

Various embodiments as set forth herein may be implemented as software (e.g., the program 140) including one or more instructions that are stored in a storage medium (e.g., the internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Herein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, methods according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., Play Store™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each element (e.g., a module or a program) of the above-described elements may include a single entity or multiple entities, and some of the multiple entities may be separately disposed in any other element. According to various embodiments, one or more of the above-described elements or operations may be omitted, or one or more other elements or operations may be added. Alternatively or additionally, a plurality of elements (e.g., modules or programs) may be integrated into a single element. In such a case, according to various embodiments, the integrated element may still perform one or more functions of each of the plurality of elements in the same or similar manner as they are performed by a corresponding one of the plurality of elements before the integration. According to various embodiments, operations performed by the module, the program, or another element may be carried out sequentially, in parallel, repeatedly, or heuristically, or one or more of the operations may be executed in a different order or omitted, or one or more other operations may be added.

What is claimed is:

1. An electronic device capable of being worn on a finger, the electronic device comprising:
a motion sensor configured to detect movement of the electronic device;
a processor; and
a memory configured to store instructions,
wherein the instructions are configured to cause, when executed by the processor, the electronic device to:
generate a first message requesting a user to perform a specified first gesture;
obtain, at least based both on the generation of the first message and data generated by the motion sensor, a first vector and a second vector indicating acceleration in a direction of gravity using;
determine a first x-axis acceleration value in an x-axis direction, a first y-axis acceleration value in a y-axis direction, and a first z-axis acceleration value in a z-axis direction using the first vector and the second vector;
determine a correction value of converting the first x-axis acceleration value, the first y-axis acceleration value, and the first z-axis acceleration value into a second x-axis acceleration value, a second y-axis acceleration value, and a second z-axis acceleration value measured when the electronic device is in a specified wearing state; and
based on the correction value, calibrate data acquired from the motion sensor.

2. The electronic device of claim 1, comprising a communication circuit configured to enable the electronic device to wirelessly communicate with an external electronic device,
wherein the instructions are configured to cause, when executed by the processor, the electronic device to:
transmit the first message to the external electronic device through the communication circuit; and
based on data generated by the motion sensor after the first message is transmitted to the external electronic device, obtain a first vector and a second vector indicating acceleration in a direction of gravity, wherein the data generated by the motion sensor after the first message is transmitted to the external electronic device is at least part of the data generated by the motion sensor.

3. The electronic device of claim 2, wherein the instructions are configured to cause, when executed by the processor, the electronic device to
retransmit the first message, based on determining that an angle between the first vector and the second vector is less than or equal to a specified threshold angle value.

4. The electronic device of claim 1, wherein the instructions are configured to cause, when executed by the processor, the electronic device to
generate the first message and obtain the first vector and the second vector, based on a sampling frequency being set to be equal to or more than a specified threshold frequency value in the motion sensor.

5. The electronic device of claim 4, wherein the instructions are configured to cause, when executed by the processor, the electronic device to, based on the sampling frequency being set to be less than the specified threshold frequency value,
generate a second message requesting the user to perform a specified second gesture;
obtain, at least based on the generation of the second message and data generated by the motion sensor, a first acceleration value when the electronic device moves in a direction opposite the direction of gravity and a second acceleration value when the electronic device moves in the direction of gravity; and
based on the first acceleration value and the second acceleration value, determine the first x-axis acceleration value, the first y-axis acceleration value in the y-axis direction, and the first z-axis acceleration value in the z-axis direction.

6. The electronic device of claim 1, wherein the instructions are configured to cause, when executed by the processor, the electronic device to:
determine a user's state based on data generated by the motion sensor; and
based on the determination of the user's state as being any of a sitting state and standing state, generate the first message and obtain the first vector and the second vector.

7. The electronic device of claim 6, wherein the instructions are configured to cause, when executed by the processor, the electronic device to, based on the determination of the user's state as being a lying down state,
generate a second message requesting the user to perform a specified second gesture;
obtain, at least based on the generation of the second message and data generated by the motion sensor, a first acceleration value when the electronic device moves in a direction opposite the direction of gravity and a second acceleration value when the electronic device moves in the direction of gravity; and
based on the first acceleration value and the second acceleration value, determine the first x-axis acceleration value, the first y-axis acceleration value in the y-axis direction, and the first z-axis acceleration value in the z-axis direction.

8. An electronic device capable of being worn on a finger, the electronic device comprising:
a communication circuit for the electronic device to wirelessly communicate with an external electronic device;
a motion sensor configured to detect movement of the electronic device;
a processor; and
a memory configured to store instructions,
wherein the instructions are configured to cause, when executed by the processor, the electronic device to:
periodically identify a user's state based on data generated by the motion sensor;
identify, based on the user's state being identified as any of a walking state and a running state and based on data generated by the motion sensor, a first direction in which a user, as wearing the electronic device, moves forward;
identify, based on data generated by the motion sensor, that the electronic device moves backward in a second direction opposite the first direction, and obtain a third acceleration value at a time at which the electronic device is determined to be moving backward;
identify, data generated by the motion sensor, that the electronic device moves forward in the first direction, and obtain a fourth acceleration value at a time at which the electronic device is determined to be moving forward;
based on the third acceleration value and the fourth acceleration value, determine a first x-axis acceleration value in an x-axis direction, a first y-axis acceleration value in a y-axis direction, and a first z-axis acceleration value in a z-axis direction;
determine a correction value of converting the first x-axis acceleration value, the first y-axis acceleration value, and the first z-axis acceleration value into a second x-axis acceleration value, a second y-axis acceleration value, and a second z-axis acceleration value measured when the electronic device is in a specified wearing state; and
based on the correction value, calibrate data acquired from the motion sensor.

9. A method for operating an electronic device capable of being worn on a finger, the method comprising:
generating a first message requesting a user to perform a specified first gesture;
obtaining, at least based both on the generation of the first message and data generated by a motion sensor of the electronic device, a first vector and a second vector indicating acceleration in a direction of gravity;
based on the first vector and the second vector, determining a first x-axis acceleration value in an x-axis direction, a first y-axis acceleration value in a y-axis direction, and a first z-axis acceleration value in a z-axis direction;
determining a correction value of converting the first x-axis acceleration value, the first y-axis acceleration value, and the first z-axis acceleration value into a second x-axis acceleration value, a second y-axis acceleration value, and a second z-axis acceleration value measured when the electronic device is in a specified wearing state; and
based on the correction value, correcting data acquired from the motion sensor.

10. The method of claim 9, wherein the electronic device comprises a communication circuit configured to enable the electronic device to wireless communication with an external electronic device,
wherein the generating comprises transmitting the first message to the external electronic device through the communication circuit,
wherein the obtaining comprises obtaining, based on data generated by the motion sensor after the first message is transmitted, the first vector and the second vector indicating acceleration in the direction of gravity,
wherein the data generated by the motion sensor after the first message is transmitted to the external electronic device is at least part of the data generated by the motion sensor.

11. The method of claim 10, further comprising retransmitting the first message, based on determining that an angle between the first vector and the second vector being is less than or equal to a specified threshold angle value.

12. The method of claim 9, wherein the generating and the obtaining are performed based on a sampling frequency being set to be equal to or more than a specified threshold frequency value in the motion sensor.

13. The method of claim 12, based on the sampling frequency being set to be less than the threshold frequency value, further comprising:
generating a second message requesting the user to perform a specified second gesture;
obtaining, at least based on the generation of the second message and data generated by the motion sensor, a first acceleration value when the electronic device moves in a direction opposite the direction of gravity and a second acceleration value when the electronic device moves in the direction of gravity; and
based on the first acceleration value and the second acceleration value, determining the first x-axis acceleration value, the first y-axis acceleration value in the y-axis direction, and the first z-axis acceleration value in the z-axis direction.

14. The method of claim 9, further comprising determining a user's state based on data generated by the motion sensor,
wherein the generating and the obtaining are performed based on determining the user's state as being any of a sitting state and a standing state.

15. The method of claim 14, based on determining the user's state as being a lying down state, further comprising:
generating a second message requesting the user to perform a specified second gesture;
obtaining, at least based both on the generation of the second message and data generated by the motion sensor, a first acceleration value when the electronic device moves in a direction opposite the direction of gravity and a second acceleration value when the electronic device moves in the direction of gravity; and
based on the first acceleration value and the second acceleration value, determining the first x-axis acceleration value, the first y-axis acceleration value in the y-axis direction, and the first z-axis acceleration value in the z-axis direction.

16. A non-transitory computer-readable recording medium storing instructions readable by a processor of an electronic device capable of being worn on a finger, wherein the instructions are configured to cause, when executed by the processor, the electronic device to perform operations of:
generating a first message requesting a user to perform a specified first gesture;
obtaining, at least based both on the generation of the first message and data generated by a motion sensor of the electronic device, a first vector and a second vector indicating acceleration in a direction of gravity;
determining a first x-axis acceleration value in an x-axis direction, a first y-axis acceleration value in a y-axis direction, and a first z-axis acceleration value in a z-axis direction using the first vector and the second vector;
determine a correction value of converting the first x-axis acceleration value, the first y-axis acceleration value, and the first z-axis acceleration value into a second x-axis acceleration value, a second y-axis acceleration value, and a second z-axis acceleration value measured when the electronic device is in a specified wearing state; and
based on the correction value, calibrating data acquired from the motion sensor.

17. The recording medium of claim 16, wherein the electronic device comprises a communication circuit configured to enable the electronic device to wireless communication with an external electronic device,
wherein the operation of generating comprises transmitting the first message to the external electronic device through the communication circuit,
wherein the operation of obtaining comprises obtaining, based on data generated by the motion sensor after the first message is transmitted, a first vector and a second vector indicating acceleration in the direction of gravity, and
wherein the data generated by the motion sensor after the first message is transmitted to the external electronic device is at least part of the data generated by the motion sensor.

18. The recording medium of claim 17, wherein the instructions are configured to cause, when executed by the processor, the electronic device to
retransmit the first message, based on determining that an angle between the first vector and the second vector is less than or equal to a specified threshold angle value.

19. The recording medium of claim 16, wherein the operation of generating and the operation of obtaining are performed based on a sampling frequency being set to be equal to or more than a specified threshold frequency value in the motion sensor.

20. The recording medium of claim 16, wherein the instructions are configured to cause, when executed by the processor, the electronic device to
perform an operation of determining a user's state based on data generated by the motion sensor, and
wherein the operation of generating and the operation of obtaining are performed based on determining the user's state as being any of a sitting state and a standing state.
